(19) United States
(12) Patent Application Publication
(10) Pub. No.: US 2025/0197437 A1
(43) Pub. Date: Jun. 19, 2025

(54) CANNABINOID COMPLEXES WITH IMPROVED PROPERTIES

(71) Applicant: KEMIJSKI INŠTITUT, Ljubljana (SI)

(72) Inventors: Mitja KRIŽMAN, Pobegi (SI); Jure ZEKIC, Ljubljana (SI); Primož ŠKET, Ljubljana (SI); Alojz ANŽLOVAR, Grosupljem (SI); Barbara ZUPANCIC, Ljubljana (SI); Jože GRDADOLNIK, Ljubljana (SI)

(73) Assignee: KEMIJSKI INŠTITUT, Ljubljana (SI)

(21) Appl. No.: 18/833,374

(22) PCT Filed: Jan. 25, 2023
(86) PCT No.: PCT/EP2023/051730

§ 371 Date: Jul. 25, 2024

(30) Foreign Application Priority Data
Jan. 25, 2022 (LU) LU501323

(51) Int. Cl. C07H 5/06; C07C 63/06

(52) U.S. Cl. CPC C07H 5/06; C07C 63/06

(57) ABSTRACT

The present invention generally relates to new cannabinoid formulations, and more specifically to complexes comprising a cannabinoid and glucosamine. The complexes of the present invention can be specifically characterized in that they have improved stability and/or solubility in water. The present invention further pertains to processes for the preparation of complexes of the present invention as well as compositions comprising them.

### CROSS REFERENCE TO RELATED APPLICATION(S)

This application is a 35 U.S.C. § 371 National Stage of International Patent Application No. PCT/EP2023/051730, filed Jan. 25, 2023, which claims priority to and the benefit of Luxembourg Patent Application No. LU501323, filed Jan. 25, 2022, the disclosure of which is incorporated herein by reference in its entirety.

### FIELD OF THE INVENTION

The present invention generally relates to new cannabinoid formulations, and more specifically to complexes comprising a cannabinoid and glucosamine. The complexes of the present invention can be specifically characterized in that they have improved stability and/or solubility in water. The present invention further pertains to processes for the preparation of complexes of the present invention as well as compositions comprising them.

### BACKGROUND OF THE INVENTION

Cannabinoids are compounds with a wide range of biological activity (Pugazhendhi et al., 2021). In nature, cannabinoids can be found in the plants of the genus Cannabis, i.e. in the plant species Cannabis sativa, Cannabis indica and Cannabis ruderalis. There are many naturally occurring cannabinoids, the most known among them are cannabinol (CBN), cannabinolic acid (CBNA), cannabidiol (CBD), cannabidiolic acid (CBDA), cannabigerol (CBG), cannabigerolic acid (CBGA), tetrahydrocannabinol (THC), tetrahydrocannabinolic acid (THCA), Δ(9)-tetrahydrocannabinol (D9THC), Δ(9)-tetrahydrocannabinolic acid (D9THCA), Δ(9)-cannabidiol (D9CBD), Δ(9)-tetrahydrocannabidiolic acid (D9THCA), Δ(8)-tetrahydrocannabinol (D8THC), Δ(8)-tetrahydrocannabinolic acid tetrahydrocannabivarin (THCV), (D8THCA), tetrahydrocannabivarinic acid (THCVA), cannabichromene (CBC), cannabichromenic acid (CBCA), cannabicyclol (CBL), cannabicyclolic acid (CBLA), cannabielsoin (CBE), cannabielsoic acid (CBEA), cannabitriol (CBT) and cannabitriolic acid (CBTA). Cannabinoids are biologically active compounds which have similar physiological activities like endogeneous cannabinoids found in humans (e.g. anandamide). Besides the naturally occuring ones, synthetic cannabinoids have also been produced. Cannabinoids have a binding affinity for endocannabinoid receptors (i.e. CB1 and CB2) and can exhibit a variety of beneficial pharmacological activities (Takeda et al., 2014).

Some cannabinoids are present in »acidic« forms, i.e. these forms are the biosynthetic precursors of decarboxylated cannabinoids. There is an ongoing scientific evidence that some acidic cannabinoids have also a pronounced biological activity, even against proliferating cancer cells. So far, the most studied acidic cannabinoid is certainly cannabidiolic acid (CBDA) (Anderson et al., 2019; Bolognini et al., 2013; Takeda et al., 2008; Takeda et al., 2014;). Other commonly known acidic cannabinoids are cannabinolic acid (CBNA), cannabigerolic acid (CBGA), tetrahydrocannabinolic acid (THCA), Δ(9)-tetrahydrocannabinolic acid (D9THCA), Δ(9)-tetrahydrocannabidiolic acid (D9THCA), Δ(8)-tetrahydrocannabinolic acid (D8THCA), tetrahydrocannabivarinic acid (THCVA), cannabichromenic acid (CBCA), cannabicyclolic acid (CBLA), cannabielsoic acid (CBEA), and cannabitriolic acid (CBTA). The main issue with acidic cannabinoids is their decarboxylation, a spontaneous, non-enzymatic process which is dependent among many environmental factors like the compound itself, temperature, light intensity, moisture, chemical composition of the medium etc. Once decarboxylated, the biological activity related to the acidic form is lost, fully or in part, depending on the decarboxylation degree.

Since cannabinoids are quite apolar compounds (i.e. lipophilic), they are usually not soluble in water media, or their water solubility is rather low. Therefore, cannabinoid preparations are traditionally lipid-based, in order to obtain a homogeneous, solubilised preparation. For this reason, cannabinoid bioavailabilty, is rather low (Izgelov et al., 2020). It is known that by increasing water solubility, bioavailability is also increased (Koch et al., 2020). An increased water solubility could also offer more options in terms of formulation composition.

Typical strategies for increased water solubility of cannabinoids are the use of micelles or emulsion (US2020/0246404 A1; US 2015/0342902 A1; US 2014/0348926 A1; WO 2018/152334), of a (nano)particle suspension (Stukelj et al., 2019), of an inclusion complex (WO 2017/183011 A1) or even polymers (Koch et al., 2020), in order to provide a rather lipophilic environment where the cannabinoids are likely to be localised in the medium.

Such strategies, despites increasing water solubility and consequentially also bioavailability, might also introduce some constraints or even issues related to their very composition, since many other ingredients are needed in the cannabinoid formulation in order to take advantage of increased water solubility.

Accordingly, it is an object of the present invention to provide a new strategy to increase water solubility of cannabinoids.

### SUMMARY OF THE INVENTION

The above objective is solved by the present invention. In the present invention, cannabinoids (in pure form or in mixture with other ingredients) are reacted with glucosamine base in solution. After evaporation of the solvent, complexes of cannabinoid(s) with glucosamine are formed. Since glucosamine interacts with the cannabinoid molecule by forming intermolecular bonds, the cannabinoid molecule then avails on the presence of a hydrophilic moiety, in this case glucosamine, for an increased solubilisation in water. Solubilisation therefore occurs without the need for the inclusion into a micelle or adsorption onto a suspended particle. A true solution is therefore the result. Additionally, due to the interaction of glucosamine with a cannabinoid molecule, including also acidic cannabinoids, a higher stability of acidic cannabinoids in solution towards decarboxylation has also been observed in the case of complexes with glucosamine. Glucosamine quickly undergoes degradation under alkaline conditions (Shu, 1998), thus it is practically not commercially available in pure basic form, but only in the form of salts.

The present invention thus provides in a further aspect a composition comprising a) a cannabinoid and b) glucosamine.

The present invention provides in a further aspect a process for the preparation of a complex according to the present invention, said process comprises the step of reacting a cannabinoid with glucosamine base.

The present invention provides in a further aspect a method for improving the solubility of a cannabinoid in water, comprising the step of reacting a cannabinoid with glucosamine base.

The present invention can be summarized by the following items.
1. Complex comprising (or consisting essentially of) a) a cannabinoid and b) glucosamine.
2. The complex according to item 1, wherein the molar ratio between the cannabinoid and glucosamine is ranging from 1:0.5 to 1:15.
3 The complex according to item 1, wherein the molar ratio between the cannabinoid and glucosamine is ranging from 1:1 to 1:10.
4. The complex according to item 1, wherein the molar ratio between the cannabinoid and glucosamine is ranging from 1:1 to 1:5.
5. The complex according to item 1, wherein the molar ratio between the cannabinoid and glucosamine is 1:1.
6. The complex according to item 1, wherein the molar ratio between the cannabinoid and glucosamine is 1:2.
7. The complex according to item 1, wherein the molar ratio between the cannabinoid and glucosamine is 1:3.
8. The complex according to item 1, wherein the molar ratio between the cannabinoid and glucosamine is 1:4.
9. The complex according to any one of items 1 to 8, wherein the cannabinoid is selected from the group consisting of: cannabinol (CBN), cannabinolic acid (CBNA), cannabidiol (CBD), cannabidiolic acid (CBDA), cannabigerol (CBG), tetrahydrocannabinol (THC), cannabigerolic acid (CBGA), tetrahydrocannabinolic acid (THCA), Δ(9)-tetrahydrocannabinol (D9THC), Δ(9)-tetrahydrocannabinolic acid (D9THCA), Δ(9)-cannabidiol (D9CBD), Δ(9)-tetrahydrocannabidiolic acid (D9THCA), Δ(8)-tetrahydrocannabinol (D8THC), Δ(8)-tetrahydrocannabinolic acid (D8THCA), tetrahydrocannabivarin (THCV), tetrahydrocannabivarin (THCV), tetrahydrocannabivarinic acid (THCVA), cannabichromene (CBC), cannabichromenic acid (CBCA), cannabicyclol (CBL), cannabicyclolic acid (CBLA), cannabielsoin (CBE), cannabielsoic acid (CBEA), cannabitriol (CBT), cannabitriolic acid (CBTA), Nabilone, and combinations thereof.
10. The complex according to any one of items 1 to 9, wherein the cannabinoid is selected from the group consisting of cannabinol, cannabinolic acid, cannabidiol (CBD), cannabidiolic acid (CBDA), cannabigerol, cannabigerolic acid, and combinations thereof.
11. The complex according to any one of items 1 to 10, wherein the cannabinoid is selected from the group consisting of cannabidiol (CBD), cannabidiolic acid (CBDA), cannabigerol (CBG), cannabigerolic acid (CBGA), and combinations thereof.
12. The complex according to any one of items 1 to 11, wherein the cannabinoid is selected from the group consisting of cannabidiol (CBD), cannabidiolic acid (CBDA), and a combination thereof.
12a. The complex according to any one of items 1 to 12, wherein the cannabinoid comprises at least one carboxylic group.
12b. The complex according to any one of items 1-12a, wherein the cannabinoid comprises at least one OH group attached to an aromatic ring.
13. The complex according to any one of items 1 to 12b, characterized in that the solubility of the cannabinoid in water is at least about 15 mg/L, such as at least about 20 mg/L, at least about 25 mg/L, at least about 30 mg/L, at least about 35 mg/L, at least about 40 mg/L, at least about 45 mg/L, at least about 50 mg/L, at least about 55 mg/L, at least about 60 mg/L, at least about 65 mg/L, at least about 70 mg/L, at least about 75 mg/L, at least about 80 mg/L, at least about 85 mg/L, at least about 90 mg/L, at least about 95 mg/L, or at least about 99 mg/L.
14. The complex according to any one of items 1 to 13, characterized in that the solubility of the cannabinoid in water is at least about 30 mg/L, such as at least about 35 mg/L, at least about 40 mg/L, at least about 45 mg/L, at least about 50 mg/L, at least about 55 mg/L, at least about 60 mg/L, at least about 65 mg/L, at least about 70 mg/L, at least about 75 mg/L, at least about 80 mg/L, at least about 85 mg/L, at least about 90 mg/L, at least about 95 mg/L, or at least about 99 mg/L.
15. The complex according to any one of items 1 to 14, characterized in that the solubility of the cannabinoid in water is at least about 30 mg/L, such as at least about 35 mg/L, at least about 40 mg/L, at least about 45 mg/L, at least about 50 mg/L, at least about 55 mg/L, at least about 60 mg/L, at least about 65 mg/L, at least about 70 mg/L, at least about 75 mg/L, at least about 80 mg/L, at least about 85 mg/L, at least about 90 mg/L, at least about 95 mg/L, or at least about 99 mg/L.
16. The complex according to any one of items 1 to 15, characterized in that the solubility of the cannabinoid in water is in the range from about 15 mg/L to about 110 mg/L, such as from about 15 mg/L to about 55 mg/L, from about 15 mg/L to about 40 mg/L from about 15 mg/L to about 35 mg/L, from about 15 mg/L to about 30 mg/L, from about 20 mg/L to about 55 mg/L, from about 20 mg/L to about 40 mg/L, from about 20 mg/L to about 35 mg/L, from about 20 mg/L to about 30 mg/L, from about 30 mg/L to about 40 mg/L, from about 30 mg/L to about 35 mg/L, from about 80 mg/L to about 110 mg/L, from about 100 mg/L to about 110 mg/L, from about 85 mg/L to about 105 mg/L or from about 80 mg/L to about 90 mg/L.
17. The complex according to any one of items 1 to 16, characterized in that the solubility of the cannabinoid in water is in the range from about 15 mg/L to about 35 mg/L, such as from about 15 mg/L to about 30 mg/L, from about 20 mg/L to about 35 mg/L, from about 20 mg/L to about 30 mg/L, or from about 30 mg/L to about 35 mg/L.
18. The complex according to any one of items 1 to 17, wherein the cannabinoid is cannabidiol (CBD).
19. The complex according to any item 18, characterized in that the solubility of the cannabinoid in water is at least about 15 mg/L, such as at least about 20 mg/L or at least about 25 mg/L.
20. The complex according to any item 18 or 19, characterized in that the solubility of the cannabinoid in water is in the range from about 15 mg/L to about 35 mg/L, such as from about 20 mg/L to about 30 mg/L.
21. The complex according to any one of items 18 to 20, having a 1H NMR (400 MHz, DMSO-d6) spectrum with characteristic peaks ranging from δ 8.672 to δ 8.676 ppm for protons of OH groups at positions 1′ and 5′.
22. The complex according to any one of items 18 to 20, having a 1H NMR (400 MHz, DMSO-d6) spectrum with characteristic peaks of δ 8.672 ppm for protons of OH groups at positions 1′ and 5′.
23. The complex according to any one of items 18 to 20, having a 1H NMR (400 MHz, DMSO-d6) spectrum with characteristic peaks of δ 8.676 ppm for protons of OH groups at positions 1′ and 5′.
24. The complex according to any one of items 18 to 20, having the following 1H NMR (400 MHz, DMSO-d6) spectrum: δ 8.672 (s, 2H), 6.01 (s, 2H), 5.07 (s, 1H), 4.48 (d, 1H), 4.40 (m, 1H), 3.82 (dm, 1H), 3.02 (m, 1H), 2.29 (t, 2H), 2.10 (m, 1H), 1.91 (m, 1H), 1.66 (m, 2H), 1.59 (s, 3H), 1.57 (s, 3H), 1.46 (m, 2H) 1.26 (m, 4H), 0.85 (t, 3H).
25. The complex according to any one of items 18 to 20, having the following 1H NMR (400 MHz, DMSO-d6) spectrum: δ 8.676 (s, 2H), 6.01 (s, 2H), 5.07 (s, 1H), 4.48 (d, 1H), 4.40 (m, 1H), 3.82 (dm, 1H), 3.02 (m, 1H), 2.29 (t, 2H), 2.10 (m, 1H), 1.91 (m, 1H), 1.66 (m, 2H), 1.59 (s, 3H), 1.57 (s, 3H), 1.46 (m, 2H) 1.26 (m, 4H), 0.85 (t, 3H).
26. The complex according to any one of items 18 to 25, having the following differences in the relative intensity, frequency and/or band shape in the Attenuated Total Reflectance Fourier-Transform Infrared (ATR-FTIR) spectrum: the bands at 3400 cm⁻¹ and 3520 cm⁻¹ have a decrease in intensity of at least about 5%, such as about 10%, and the band at 2750 cm⁻¹ has an increase in intensity of at least about 5%, such as about 10%, in comparison to the physical mixture.
27. The complex according to any one of items 18 to 26, having a transition temperature in the range from about 62 to about 70° C., such as from 65.8 to 66.4° C., during first heating, and having an enthalpy from about 15 to about 60 J/g, such as from about 19.8 to about 48.3 J/g, during first heating (as measured by DSC).
28. The complex according to any one of items 18 to 27, having a transition temperature in the range from about 62 to about 70° C., such as about 65.8° C., during first heating (as measured by DSC).
29. The complex according to any one of items 18 to 28, having a transition enthalpy in the range from about 30 to about 50 J/g, such as about 38.7 J/g.
30. The complex according to any one of items 18 to 27, having a transition temperature in the range from about 62 to about 70° C., such as about 66.0° C., during first heating (as measured by DSC).
31. The complex according to any one of items 18 to 27 and 30, having a transition enthalpy in the range from about 40 to about 60 J/g, such as about 48.3 J/g.
32. The complex according to any one of items 18 to 27, having a transition temperature in the range from about 62 to about 70° C., such as about 66.4° C., during first heating (as measured by DSC).
33. The complex according to any one of items 18 to 27 and 32, having a transition enthalpy in the range from about 15 to about 25 J/g, such as about 19.8 J/g.
34. The complex according to any one of items 1 to 17, wherein the cannabinoid is cannabidiolic acid (CBDA).
35. The complex according to item 34, characterized in that the solubility of the cannabinoid in water is at least about 30 mg/L or at least about 75 mg/L.
36. The complex according to item 34 or 35, characterized in that the solubility of the cannabinoid in water is in the range from about 30 mg/L to about 35 mg/L or from about 80 mg/L to about 110, such as from about 85 mg/L to about 105 mg/L.
37. The complex according to any one of items 34 to 36, characterized in that the stability of the cannabinoid in water is increased by at least about 10%, such as at least about 20%, after heating at 60° C. for 3 days compared to the stability of the cannabinoid in the absence of glucosamine under comparable conditions.
38. The complex according to any one of items 34 to 37, having a 1H NMR (400 MHz, DMSO-d6) spectrum with a characteristic peak of from δ 15.1 to 0 16.7 ppm for proton of OH group at position 1′.
39. The complex according to any one of items 34 to 37, having a 1H NMR (400 MHz, DMSO-d6) spectrum with a characteristic peak of δ 15.1 ppm for proton of OH group at position 1′.
40. The complex according to any one of items 34 to 37, having a 1H NMR (400 MHz, DMSO-d6) spectrum with a characteristic peak of δ 16.5 ppm for proton of OH group at position 1′.
41. The complex according to any one of items 34 to 37, having a 1H NMR (400 MHz, DMSO-d6) spectrum with a characteristic peak of δ 16.7 ppm for proton of OH group at position 1′.
42. The complex according to any one of items 34 to 41, having a 1H NMR (400 MHz, DMSO-d6) spectrum with a characteristic peak of from δ 5.84 to 6.01 ppm for proton at position 4′.
43. The complex according to any one of items 34 to 41, having a 1H NMR (400 MHz, DMSO-d6) spectrum with a characteristic peak of δ 5.84 ppm for proton at position 4′.
44. The complex according to any one of items 34 to 41, having a 1H NMR (400 MHz, DMSO-d6) spectrum with a characteristic peak of δ 5.86 ppm for proton at position 4′.
45. The complex according to any one of items 34 to 44, having a 1H NMR (400 MHz, DMSO-d6) spectrum with a characteristic peak of δ 6.01 ppm for proton at position 4′.
46. The complex according to any one of items 34 to 45, having a 1H NMR (400 MHz, DMSO-d6) spectrum with a characteristic peak of from δ 8.73 to 9.08 ppm for proton of OH group at position 5′.
47. The complex according to any one of items 34 to 45, having a 1H NMR (400 MHz, DMSO-d6) spectrum with a characteristic peak of δ 8.73 ppm for proton of OH group at position 5′.
48. The complex according to any one of items 34 to 45, having a 1H NMR (400 MHz, DMSO-d6) spectrum with a characteristic peak of δ 8.80 ppm for proton of OH group at position 5′.
49. The complex according to any one of items 34 to 45, having a 1H NMR (400 MHz, DMSO-d6) spectrum with a characteristic peak of δ 9.08 ppm for proton of OH group at position 5′.
50. The complex according to any one of items 38 to 49, wherein in the 1H NMR spectrum the signal for 2′ COOH proton is not present.
51. The complex according to any one of items 34 to 37, having the following 1H NMR (400 MHz, DMSO-d6) spectrum: δ 15.1 (s, 1H), 9.08 (s, 1H), 5.94 (s, 1H), 5.06 (s, 1H), 4.48 (d, 1H), 4.39 (m, 1H), 3.87 (m, 1H), 2.09 (m, 1H), 1.91 (m, 1H), 1.67 (m, 2H), 1.59 (s, 3H), 1.58 (s, 3H), 1.45 (m, 2H) 1.25 (m, 4H), 0.85 (t, 3H).
52. The complex according to any one of items 34 to 37, having the following 1H NMR (400 MHz, DMSO-d6) spectrum: δ 16.5 (s, 1H), 8.80 (s, 1H), 5.86 (s, 1H), 5.05 (s, 1H), 4.49 (d, 1H), 4.38 (m, 1H), 3.86 (m, 1H), 2.09 (m, 1H), 1.90 (m, 1H), 1.66 (m, 2H), 1.58 (s, 3H), 1.58 (s, 3H), 1.44 (m, 2H) 1.25 (m, 4H), 0.85 (t, 3H).
53. The complex according to any one of items 34 to 37, having the following 1H NMR (400 MHz, DMSO-d6) spectrum: δ 16.7 (s, 1H), 8.73 (s, 1H), 5.84 (s, 1H), 5.05 (s, 1H), 4.49 (d, 1H), 4.38 (m, 1H), 3.86 (m, 1H), 2.09 (m, 1H), 1.90 (m, 1H), 1.66 (m, 2H), 1.58 (s, 3H), 1.58 (s, 3H), 1.44 (m, 2H) 1.25 (m, 4H), 0.85 (t, 3H).
54. The complex according to any one of items 34 to 53, having the following differences in the relative intensity, frequency and/or band shape in the Attenuated Total Reflectance Fourier-Transform Infrared (ATR-FTIR) spectrum: the band at 3300 cm⁻¹ has a decrease in intensity of at least about 15%, such as about 25%, the band at 1372 cm⁻¹ has an increase in intensity of at least about 80%, such as about 95%, and the band at 2300 cm⁻¹ has an increase in intensity of at least about 20%, such as about 33%, in comparison to the physical mixture.
55. The complex according to any one of items 34 to 54, having a first transition temperature in the range from about 30° C. to about 100° C., such as about 73.6° C., a second transition temperature in the range from about 100° C. to about 106° C., such as about 104.6° C., and a third transition temperature in the range from about 106° C. to about 110° C., such as about 107.4° C., during first heating (as measured by DSC).
56. The complex according to any one of items 34 to 55, having a first transition enthalpy in the range from about 10 to about 50 J/g, such as about 38.4 J/g, a second transition enthalpy in the range from about 0.1 to about 1.0 J/g, such as about 0.3 J/g, and a third transition enthalpy in the range from about 0.1 to about 1.0 J/g, such as about 0.4 J/g, during first heating (as measured by DSC).
57. The complex according to any one of items 1 to 17, wherein the cannabinoid is cannabigerol (CBG).
58. The complex according to any item 57, characterized in that the solubility of the cannabinoid in water is at least about 15 mg/L, such as at least about 20 mg/L, at least about 25 mg/L, at least about 27 mg/L, or at least about 30 mg/L.
59. The complex according to any item 57 or 58, characterized in that the solubility of the cannabinoid in water is in the range from about 15 mg/L to about 35 mg/L, such as from about 18 mg/L to about 32 mg/L or from about 20 mg/L to about 30 mg/L.
60. The complex according to any one of items 57 to 59, having a 1H NMR (400 MHz, DMSO-d6) spectrum with characteristic peaks of δ 8.89 ppm for protons of OH groups at positions 1 and 5.
61. The complex according to any one of items 57 to 60, having the following 1H NMR (400 MHz, DMSO-d6) spectrum: δ 8.89 (s, 2H), 6.08 (s, 2H), 5.15 (m, 1H), 5.04 (m, 1H), 3.11 (d, 2H), 1.97 (m, 2H), 1.87 (m, 2H), 1.68 (s, 3H), 1.60 (s, 3H), 1,52 (s, 3H), 1.46 (m, 2H), 1.26 (m, 4H), 0.85 (t, 3H).
62. The complex according to any one of items 57 to 61, having the following differences in the relative intensity, frequency and/or band shape in the Attenuated Total Reflectance Fourier-Transform Infrared (ATR-FTIR) spectrum: the band at 2750 cm⁻¹ has a decrease in intensity of at least about 10%, such as about 15%, and the band at 3300 cm⁻¹ has an increase in intensity of at least about 10%, such as about 15%, in comparison to the physical mixture.
63. The complex according to any one of items 57 to 62, having a transition temperature in the range from about 40° C. to about 70° C., such as about 53.1° C., during first heating (as measured by DSC).
64. The complex according to any one of items 57 to 63, having a transition enthalpy in the range from about 30 to about 70 J/g, such as about 51.2 J/g, during first heating (as measured by DSC).
64a. The complex according to any one of items 1 to 64, which is an isolated complex.
65. The complex according to any one of items 1 to 64a, obtainable by a process according to any one of items 74 to 80.
66. A composition comprising a) a cannabinoid and b) glucosamine.
67. The composition according to item 66, characterized in that the solubility of the cannabinoid in water is at least about 15 mg/L, such as at least about 20 mg/L, at least about 25 mg/L, at least about 30 mg/L, at least about 35 mg/L, at least about 40 mg/L, at least about 45 mg/L, at least about 50 mg/L, at least about 55 mg/L, at least about 60 mg/L, at least about 65 mg/L, at least about 70 mg/L, at least about 75 mg/L, at least about 80 mg/L, at least about 85 mg/L, at least about 90 mg/L, at least about 95 mg/L, or at least about 99 mg/L.
68. The composition according to item 66 or 67, characterized in that the solubility of the cannabinoid in water is at least about 25 mg/L, such as at least about 30mg/L, at least about 35 mg/L, at least about 40 mg/L, at least about 45 mg/L, at least about 50 mg/L, at least about 55 mg/L, at least about 60 mg/L, at least about 65 mg/L, at least about 70 mg/L, at least about 75 mg/L, at least about 80 mg/L, at least about 85 mg/L, at least about 90 mg/L, at least about 95 mg/L, or at least about 99 mg/L.
69. The composition according to any one of items 66 to 68, characterized in that the solubility of the cannabinoid in water is at least about 30 mg/L, such as at least about 35 mg/L, at least about 40 mg/L, at least about 45 mg/L, at least about 50 mg/L, at least about 55 mg/L, at least about 60 mg/L, at least about 65 mg/L, at least about 70 mg/L, at least about 75 mg/L, at least about 80 mg/L, at least about 85 mg/L, at least about 90 mg/L, at least about 95 mg/L, or at least about 99 mg/L.
70. The composition according to any one of items 66 to 69, characterized in that the solubility of the cannabinoid in water is in the range from about 15 mg/L to about 110 mg/L, such as from about 15 mg/L to about 55 mg/L, from about 15 mg/L to about 40 mg/L from about 15 mg/L to about 35 mg/L, from about 15 mg/L to about 30 mg/L, from about 20 mg/L to about 55 mg/L, from about 20 mg/L to about 40 mg/L, from about 20 mg/L to about 35 mg/L, from about 20 mg/L to about 30 mg/L, from about 30 mg/L to about 40 mg/L, from about 30 mg/L to about 35 mg/L, from about 80 mg/L to about 110 mg/L, from about 100 mg/L to about 110 mg/L, from about 85 mg/L to about 105 mg/L or from about 80 mg/L to about 90 mg/L.
71. The composition according to any one of items 66 to 70, characterized in that the solubility of the cannabinoid in water is in the range from about 15 mg/L to about 35 mg/L, such as from about 15 mg/L to about 30 mg/L, from about 20 mg/L to about 35 mg/L, from about 20 mg/L to about 30 mg/L, or from about 30 mg/L to about 35 mg/L.
72. The composition according to item 66, comprising at least one complex according to any one of items 1 to 65.
72a. The composition according to item 72, wherein said isolated complex comprises at least 30%, at least 40% at least 50%, at least 60%, at least 75% or at least 90% of the total cannabinoid in a form complexed with glucosamine.
73. The composition according to any one of items 66 to 72a, wherein the composition comprises a hemp extract.
74. Process for the preparation of a complex according to any one of items 1 to 65, said process comprises the step of reacting the cannabinoid with glucosamine base.
75. The process according to item 74, wherein the reaction is carried out in one or more suitable solvents which dissolve(s) both compounds (e.g. methanol, ethanol etc) or a mixture of solvents (e.g., 50% ethanol).
76. The process according to item 74 or 75, comprising a pre-treatment step which comprises stripping off the counter ion (i.e. anion, usually sulfate or chloride) from glucosamine in salt form in order to obtain said glucosamine base.
77. The process according to item 76, wherein the stripping off is facilitated by use of an appropriate means, such as by anion-exchange chromatography.
78. The process according to item 76 or 77, wherein the pre-treatment step further comprises drying the obtained glucosamine base (e.g., by evaporation or lyophilisation).
79. The process according to any one of items 74 to 78, wherein the step of reacting the cannabinoid with glucosamine base is carried out at a temperature ranging from about 10° C. to about 60° C.
80. The process according to any one of items 74 to 79, where in the step of reacting the cannabinoid with glucosamine base is carried out for a time period from about 2 minutes to about 2 hours.
80a. The process according to any one of items 74 to 80 wherein the step of reacting the cannabinoid with glucosamine base is followed by a concentration step, e.g. evaporation of solvent.
81. Method for improving the solubility of a cannabinoid in water, comprising the step of reacting the cannabinoid with glucosamine base.
82. The method according to item 81, wherein the reaction is carried out in one or more suitable solvents which dissolve(s) both compounds (e.g. methanol, ethanol etc) or a mixture of solvents (e.g., 50% ethanol).
83. The method according to item 81 or 82, comprising a pre-treatment step which comprises stripping off the counter ion (i.e. anion, usually sulfate or chloride) from glucosamine in salt form in order to obtain said glucosamine base.
84. The method according to item 83, wherein the stripping off is facilitated by use of an appropriate means, such as by anion-exchange chromatography.
85. The method according to item 83 or 84, wherein the pre-treatment step further comprises drying the obtained glucosamine base (e.g., by evaporation or lyophilisation).
86. The methods according to any one of items 81 to 85, wherein the step of reacting the cannabinoid with glucosamine base is carried out at a temperature ranging from about 10° C. to about 60° C.
87. The method according to any one of items 81 to 86, where in the step of reacting the cannabinoid with glucosamine base is carried out for a time period from about 2 minutes to about 2 hours.
87a. The method according to any one of items 81 to 87 wherein the step of reacting the cannabinoid with glucosamine base is followed by a concentration step, e.g. evaporation of solvent.
88. The method according to any one of items 81 to 87, wherein the cannabinoid is selected from the group consisting of: cannabinol (CBN), cannabinolic acid (CBNA), cannabidiol (CBD), cannabidiolic acid (CBDA), cannabigerol (CBG), cannabigerolic acid (CBGA), tetrahydrocannabinol (THC), tetrahydrocannabinolic acid (THCA), Δ(9)-tetrahydrocannabinol (D9THC), Δ(9)-tetrahydrocannabinolic acid (D9THCA), Δ(9)-cannabidiol (D9CBD), Δ(9)-tetrahydrocannabidiolic acid (D9THCA), Δ(8)-tetrahydrocannabinol (D8THC), Δ(8)-tetrahydrocannabinolic acid (D8THCA), tetrahydrocannabivarin (THCV), tetrahydrocannabivarin (THCV), tetrahydrocannabivarinic acid (THCVA), cannabichromene (CBC), cannabichromenic acid (CBCA), cannabicyclol (CBL), cannabicyclolic acid (CBLA), cannabielsoin (CBE), cannabielsoic acid (CBEA), cannabitriol (CBT), cannabitriolic acid (CBTA), Nabilone, and combinations thereof.
89. The method according to any one of items 81 to 88, wherein the cannabinoid is selected from the group consisting of cannabinol, cannabinolic acid, cannabidiol (CBD), cannabidiolic acid (CBDA), cannabigerol, cannabigerolic acid, and combinations thereof.
90. The method according to any one of items 81 to 89, wherein the cannabinoid is selected from the group consisting of cannabidiol (CBD), cannabidiolic acid (CBDA), cannabigerol (CBG), cannabigerolic acid (CBGA), and combinations thereof.
91. The method according to any one of items 81 to 90, wherein the cannabinoid is selected from the group consisting of cannabidiol (CBD), cannabidiolic acid (CBDA), and a combination thereof.
92. The method according to any one of items 81 to 90, wherein the cannabinoid is cannabidiol (CBD).
93. The method according to any one of items 81 to 90, wherein the cannabinoid is cannabidiolic acid (CBDA).
94. The method according to any one of items 81 to 90, wherein the cannabinoid is selected from the group consisting of cannabigerol (CBG), cannabigerolic acid (CBGA) and a combination thereof.
95. The method according to any one of items 81 to 90, wherein the cannabinoid is cannabigerol (CBG).
96. The method according to any one of items 81 to 95, wherein the cannabinoid is cannabigerolic acid (CBGA).
97. The method according to any one of items 81 to 96, wherein the cannabinoid comprises at least one carboxylic group.
98. The method according to any one of items 81-97, wherein the cannabinoid comprises at least one OH group attached to an aromatic ring.
99. Use of glucosamine base for improving the solubility and/or stability of a cannabinoid in water.

### BRIEF DESCRIPTION OF THE DRAWINGS

FIG. 1: Determined concentrations of CBD, CBDA and CBG in water solution (CBD is depicted as left bar, CBG as middle bar and CBDAas right bar for each ratio). For the test, pure CBD, CBDA and CBG as well as CBD:GA, CBDA:GA and CBG:GA complexes in different ratios were applied.

FIG. 2: Determined concentrations of cannabinoids from hemp resin in water solution. For the test, hemp resin and GA modified resins in different molar ratios were applied.

FIG. 3: Stability of CBDA in water solution (from non-modified hemp extract and extract modified with glucosamine in ratio CBDA:glucosamine 1:2)

FIG. 4: DSC measurement of CBD:GA mixture 1:1

FIG. 5: DSC measurement of CBD:GA complex 1:1

FIG. 6: DSC measurement of CBD:GA mixture 1:2

FIG. 7: DSC measurement of CBD:GA complex 1:2

FIG. 8: DSC measurement of CBD:GA mixture 1:3

FIG. 9: DSC measurement of CBD:GA complex 1:3

FIG. 10: DSC measurement of CBG:GA mixture 1:2

FIG. 11: DSC measurement of CBG:GA complex 1:2

FIG. 12: ATR spectrum of CBDA-GA mixture (A) and CBDA-GA complex (B)

FIG. 13: ATR spectrum of CBD-GA mixture (A) and CBD-GA complex (B)

FIG. 14: ATR spectrum of CBG-GA mixture (A) and CBG-GA complex (B)

FIG. 15: Chromatograms of the separation of CBD-GA complex and CBD-GA mixture from preparation made with glucosamine base (dotted line) and with glucosamine hydrochloride (solid line). Measured values are concentration (CBD, with GA base)=32 ppm and concentration (CBD, with GA.HCl) is below level of quantification.

FIG. 16: Chromatograms of the separation of CBG-GA complex and CBG-GA mixture from preparation made with glucosamine base (dotted line) and with glucosamine hydrochloride (solid line). Measured values are concentration (CBG, with GA base)=31 ppm and concentration (CBG, with GA.HCl) is below level of quantification.

FIG. 17: Chromatograms of the separation of CBDA-GA complex and CBDA-GA mixture from preparation made with glucosamine base (dotted line) and with glucosamine hydrochloride (solid line). Measured values are concentration (CBDA, with GA base)=86 ppm and concentration (CBDA, with GA.HCl)=7 ppm (in the concentration range of unmodified CBDA).

### DETAILED DESCRIPTION OF THE INVENTION

As noted above, the present invention is based on the surprising finding that reacting a cannabinoid with a glucosamine base results in complexes which show improved stability and/or solubility in water.

The present invention thus provides in a first aspect a complex comprising (or consisting essentially of) a) a cannabinoid and b) glucosamine.

At a molecular level, the complex may have any desirable molar ratio between the cannabinoid and glucosamine, but preferably the molar ratio between the cannabinoid and glucosamine is generally ranging from 1:0.5 to 1:15 (cannabinoid:glucosamine).

According to some embodiments, molar ratio between the cannabinoid and glucosamine is ranging from 1:1 to 1:10 (cannabinoid:glucosamine).

According to some embodiments, molar ratio between the cannabinoid and glucosamine is ranging from 1:1 to 1:5 (cannabinoid:glucosamine).

According to some embodiments, molar ratio between the cannabinoid and glucosamine is ranging from 1:1 to 1:4 (cannabinoid:glucosamine).

According to some embodiments, molar ratio between the cannabinoid and glucosamine is ranging from 1:1 to 1:3 (cannabinoid:glucosamine).

According to some embodiments, the molar ratio between the cannabinoid and glucosamine is 1:1 (cannabinoid:glucosamine).

According to some embodiments, the molar ratio between the cannabinoid and glucosamine is 1:2 (cannabinoid:glucosamine).

According to some embodiments, the molar ratio between the cannabinoid and glucosamine is 1:3 (cannabinoid:glucosamine).

According to some embodiments, the molar ratio between the cannabinoid and glucosamine is 1:4 (cannabinoid:glucosamine).

According to some embodiments, the complex of the invention is characterized in that the solubility of the cannabinoid in water is at least about 15 mg/L, such as at least about 20 mg/L, at least about 25 mg/L, at least about 30 mg/L, at least about 35 mg/L, at least about 40 mg/L, at least about 45 mg/L, at least about 50 mg/L, at least about 55 mg/L, at least about 60 mg/L, at least about 65 mg/L, at least about 70 mg/L, at least about 75 mg/L, at least about 80 mg/L, at least about 85 mg/L, at least about 90 mg/L, at least about 95 mg/L, or at least about 99 mg/L.

According to some embodiments, the complex of the invention is characterized in that the solubility of the cannabinoid in water is at least about 25 mg/L, such as at least about 30 mg/L, at least about 35 mg/L, at least about 40 mg/L, at least about 45 mg/L, at least about 50 mg/L, at least about 55 mg/L, at least about 60 mg/L, at least about 65 mg/L, at least about 70 mg/L, at least about 75 mg/L, at least about 80 mg/L, at least about 85 mg/L, at least about 90 mg/L, at least about 95 mg/L, or at least about 99 mg/L.

According to some embodiments, the complex of the invention is characterized in that the solubility of the cannabinoid in water is at least about 30 mg/L, such as at least about 35 mg/L, at least about 40 mg/L, at least about 45 mg/L, at least about 50 mg/L, at least about 55 mg/L, at least about 60 mg/L, at least about 65 mg/L, at least about 70 mg/L, at least about 75 mg/L, at least about 80 mg/L, at least about 85 mg/L, at least about 90 mg/L, at least about 95 mg/L, or at least about 99 mg/L.

According to some embodiments, the complex of the invention is characterized in that the solubility of the cannabinoid in water is at least about 50 mg/L, such as at least about 55 mg/L, at least about 60 mg/L, at least about 65 mg/L, at least about 70 mg/L, at least about 75 mg/L, at least about 80 mg/L, at least about 85 mg/L, at least about 90 mg/L, at least about 95 mg/L, or at least about 99 mg/L.

According to some embodiments, the complex of the invention is characterized in that the solubility of the cannabinoid in water is at least about at least about 75 mg/L, such as at least about 80 mg/L, at least about 90 mg/L, at least about 95 mg/L, or at least about 99 mg/L.

According to some embodiments, the complex of the invention is characterized in that the solubility of the cannabinoid in water is in the range from about 15 mg/L to about 110 mg/L, such as from about 15 mg/L to about 55 mg/L, from about 15 mg/L to about 40 mg/L from about 15 mg/L to about 35 mg/L, from about 15 mg/L to about 30 mg/L, from about 20 mg/L to about 55 mg/L, from about 20 mg/L to about 40 mg/L, from about 20 mg/L to about 35 mg/L, from about 20 mg/L to about 30 mg/L, from about 30 mg/L to about 40 mg/L, from about 30 mg/L to about 35 mg/L, from about 80 mg/L to about 110 mg/L, from about 100 mg/L to about 110 mg/L, from about 85 mg/L to about 105 mg/L or from about 80 mg/L to about 90 mg/L.

According to some embodiments, the complex of the invention is characterized in that the solubility of the cannabinoid in water is in the range from about 15 mg/L to about 35 mg/L, such as from about 15 mg/L to about 30 mg/L, from about 20 mg/L to about 35 mg/L, from about 20 mg/L to about 30 mg/L, or from about 30 mg/L to about 35 mg/L.

According to some embodiments, the complex of the invention is characterized in that the solubility of the cannabinoid in water is in the range from about 15 mg/L to about 30 mg/L.

According to some embodiments, the complex of the invention is characterized in that the solubility of the cannabinoid in water is in the range from about 15 mg/L to about 20 mg/L.

According to some embodiments, the complex of the invention is characterized in that the solubility of the cannabinoid in water is in the range from about 18 mg/L to about 22 mg/L.

According to some embodiments, the complex of the invention is characterized in that the solubility of the cannabinoid in water is in the range about 20 mg/L to about 35 mg/L.

According to some embodiments, the complex of the invention is characterized in that the solubility of the cannabinoid in water is in the range about 20 mg/L to about 30 mg/L.

According to some embodiments, the complex of the invention is characterized in that the solubility of the cannabinoid in water is in the range about 20 mg/L to about 25 mg/L.

According to some embodiments, the complex of the invention is characterized in that the solubility of the cannabinoid in water is in the range about 25 mg/L to about 30 mg/L.

According to some embodiments, the complex of the invention is characterized in that the solubility of the cannabinoid in water is in the range from about 80 mg/L to about 110 mg/L.

According to some embodiments, the complex of the invention is characterized in that the solubility of the cannabinoid in water is in the range from about 85 mg/L to about 105 mg/L.

According to some embodiments, the complex of the invention is characterized in that the solubility of the cannabinoid in water is in the range from about 80 mg/L to about 90 mg/L.

According to some embodiments, the complex of the invention is characterized in that the solubility of the cannabinoid in water is in the range from about 100 mg/L to about 105 mg/L.

Water solubility may be determined by the following test: Water solubility studies are done by mixing 1 to 1.5 mg of the prepared sample with 1 mL of distilled water and stirring the mixture for 5-10 min in a 2 mL centrifuge tube on a vortex mixer. If considered necessary, sonication can also be applied. Afterwards, the solution is centrifuged at 3.000 g for 3 minutes and the supernatant is transferred to a HPLC vial. Concentration of dissolved cannabinoids is determined by analysis on an Agilent 1260 Infinity (Santa Clara, CA, USA) HPLC system. The chromatographic method is adapted from the literature (Križman, 2019). In short, the separation is performed on a Phenomenex (Torrance, CA, USA) Luna C18 (2) (octadecyl silica) chromatographic column, with dimensions 150 mm×3 mm i.d., 3 μm particle size, and temperature set at 37° C. Mobile phase is isocratic consisting of water/acetonitrile with a ratio of 9:31 (v/v), with 0.1% formic acid (v/v) and 10 mM ammonium formate, at a flow rate of 0.8 mL/min and injection volume 5 μL. The UV detection wavelength is 275 nm. The concentration of cannabinoids is determined using an external standard method.

The cannabinoid component may comprise a single cannabinoid compound or a plurality of cannabinoid compounds, either in substantially pure form, or mixed with various other compounds. The cannabinoid component may be isolated or purified from a natural source such as a cannabis plant, or a chemically-synthesized cannabinoid compound. The cannabinoid component can include, but is not limited to, cannabinoid compounds that may naturally occur in different combinations and relative quantities in the plant tissues of various species, subspecies, hybrids, strains, chemovars, and other genetic variants of the genus Cannabis, including material that may variously be classified as “marijuana” and “hemp” in accordance with various legal or technical definitions and standards. The cannabinoid component can comprise both decarboxylated cannabinoid compounds as well as the corresponding carboxylic acid forms, such as, for example, both CBD and CBDA. A cannabis extract or cannabinoid component can be decarboxylated, such as by heating.

According to some embodiments, the cannabinoid is selected from the group consisting of: cannabinol (CBN), cannabinolic acid (CBNA), cannabidiol (CBD), cannabidiolic acid (CBDA), cannabigerol (CBG), cannabigerolic acid (CBGA), tetrahydrocannabinol (THC), tetrahydrocannabinolic acid (THCA), Δ(9)-tetrahydrocannabinol (D9THC), Δ(9)-tetrahydrocannabinolic acid (D9THCA), Δ(9)-cannabidiol (D9CBD), Δ(9)-tetrahydrocannabidiolic acid (D9THCA), Δ(8)-tetrahydrocannabinol (D8THC), Δ(8)-tetrahydrocannabinolic acid (D8THCA), tetrahydrocannabivarin (THCV), tetrahydrocannabivarin (THCV), tetrahydrocannabivarinic acid (THCVA), cannabichromene (CBC), cannabichromenic acid (CBCA), cannabicyclol (CBL), cannabicyclolic acid (CBLA), cannabielsoin (CBE), cannabielsoic acid (CBEA), cannabitriol (CBT), cannabitriolic acid (CBTA), Nabilone, and combinations thereof.

According to some embodiments, the cannabinoid is selected from the group consisting of cannabinol, cannabinolic acid, cannabidiol (CBD), cannabidiolic acid (CBDA), cannabigerol, cannabigerolic acid, and combinations thereof.

According to some embodiments, the cannabinoid is selected from the group consisting of cannabidiol (CBD), cannabidiolic acid (CBDA), cannabigerol (CBG), cannabigerolic acid (CBGA), and combinations thereof.

According to some embodiments, the cannabinoid is selected from the group consisting of cannabidiol (CBD), cannabidiolic acid (CBDA), and a combination thereof.

According to some embodiments, the cannabinoid is cannabidiol (CBD).

According to some embodiments, the cannabidiol containing complex is characterized in that the solubility of the cannabinoid in water is at least about 15 mg/L, such as at least about 20 mg/L, at least about 25 mg/L, at least about 30 mg/L, at least about 35 mg/L, at least about 40 mg/L, at least about 45 mg/L, at least about 50 mg/L, at least about 55 mg/L, at least about 60 mg/L, at least about 65 mg/L, at least about 70 mg/L, at least about 75 mg/L, at least about 80 mg/L, at least about 85 mg/L, at least about 90 mg/L, at least about 95 mg/L, or at least about 99 mg/L.

According to some embodiments, the cannabidiol containing complex is characterized in that the solubility of the cannabinoid in water is at least about 25 mg/L, such as at least about 30 mg/L, at least about 35 mg/L, at least about 40 mg/L, at least 45 mg/L, at least about 50 mg/L, at least about 55 mg/L, at least about 60 mg/L, at least about 65 mg/L, at least about 70 mg/L, at least about 75 mg/L, at least about 80 mg/L, at least about 85 mg/L, at least about 90 mg/L, at least about 95 mg/L, or at least about 99 mg/L.

According to some embodiments, the cannabidiol containing complex is characterized in that the solubility of the cannabinoid in water is in the range from about 15 mg/L to about 35 mg/L, such as from about 20 mg/L to about 30 mg/L.

According to some embodiments, the cannabidiol containing complex is characterized in that the solubility of the cannabinoid in water is in the range from about 20 mg/L to about 30 mg/L, such as from about 20 mg/L to about 25 mg/L.

According to some embodiments, the cannabidiol containing complex is characterized in that the solubility of the cannabinoid in water is in the range from about 20 mg/L to about 25 mg/L, such as from about 20 mg/L to about 22 mg/L.

According to some embodiments, the cannabidiol containing complex is characterized in that the solubility of the cannabinoid in water is in the range from about 25 mg/L to about 30 mg/L, such as from about 26 mg/L to about 29 mg/L.

According to some embodiments, the cannabidiol containing complex has a 1H NMR (400 MHz, DMSO-d6) spectrum with characteristic peaks ranging from δ 8.672 to δ 8.676 ppm for protons of OH groups at positions 1′ and 5′.

According to some embodiments, the cannabidiol containing complex has a 1H NMR (400 MHz, DMSO-d6) spectrum with characteristic peaks of δ 8.672 ppm for protons of OH groups at positions 1′ and 5′.

According to some embodiments, the cannabidiol containing complex has a 1H NMR (400 MHz, DMSO-d6) spectrum with characteristic peaks of δ 8.676 ppm for protons of OH groups at positions 1′ and 5′.

According to some embodiments, the cannabidiol containing complex has the following 1H NMR (400 MHz, DMSO-d6) spectrum: δ 8.672 (s, 2H), 6.01 (s, 2H), 5.07 (s, 1H), 4.48 (d, 1H), 4.40 (m, 1H), 3.82 (dm, 1H), 3.02 (m, 1H), 2.29 (t, 2H), 2.10 (m, 1H), 1.91 (m, 1H), 1.66 (m, 2H), 1.59 (s, 3H), 1.57 (s, 3H), 1.46 (m, 2H) 1.26 (m, 4H), 0.85 (t, 3H).

According to some embodiments, the cannabidiol containing complex has the following 1H NMR (400 MHz, DMSO-d6) spectrum: δ 8.676 (s, 2H), 6.01 (s, 2H), 5.07 (s, 1H), 4.48 (d, 1H), 4.40 (m, 1H), 3.82 (dm, 1H), 3.02 (m, 1H), 2.29 (t, 2H), 2.10 (m, 1H), 1.91 (m, 1H), 1.66 (m, 2H), 1.59 (s, 3H), 1.57 (s, 3H), 1.46 (m, 2H) 1.26 (m, 4H), 0.85 (t, 3H).

According to some embodiments, the cannabidiol containing complex has the following differences in the relative intensity, frequency and/or band shape in the Attenuated Total Reflectance Fourier-Transform Infrared (ATR-FTIR) spectrum: the bands at 3400 cm⁻¹ and 3520 cm⁻¹ have a decrease in intensity of at least about 5%, such as about 10%, and the band at 2750 cm⁻¹ has an increase in intensity of at least about 5%, such as about 10%, in comparison to the physical mixture.

According to some embodiments, the cannabidiol containing complex has a transition temperature in the range from about 62 to about 70° C., such as from about 65.8 to about 66.4° C. during first heating (as measured by DSC).

According to some embodiments, the cannabidiol containing complex has a transition temperature in the range from about 65.8 to about 66.4° C. during first heating (as measured by DSC).

According to some embodiments, the cannabidiol containing complex has a transition temperature of about 65.8° C. during first heating (as measured by DSC).

According to some embodiments, the cannabidiol containing complex has a transition temperature of about 66.0° C. during first heating (as measured by DSC).

According to some embodiments, the cannabidiol containing complex has a transition temperature of about 66.4° C. during first heating (as measured by DSC).

According to some embodiments, the cannabidiol containing complex has an enthalpy from about 15 to about 60 J/g, such as from about 19.8 to about 48.3 J/g, during first heating (as measured by DSC).

According to some embodiments, the cannabidiol containing complex has an enthalpy from about 30 to about 60 J/g.

According to some embodiments, the cannabidiol containing complex has an enthalpy from about 40 to about 60 J/g.

According to some embodiments, the cannabidiol containing complex has an enthalpy from about 30 to about 50 J/g.

According to some embodiments, the cannabidiol containing complex has an enthalpy from about 30 to about 50 J/g.

According to some embodiments, the cannabidiol containing complex has an enthalpy from about 30 to about 40 J/g.

According to some embodiments, the cannabidiol containing complex has an enthalpy from about 40 to about 50 J/g.

According to some embodiments, the cannabidiol containing complex has an enthalpy from about 15 to about 30 J/g.

According to some embodiments, the cannabidiol containing complex has an enthalpy from about 15 to about 25 J/g.

According to some embodiments, the cannabidiol containing complex has an enthalpy of about 38.7 J/g.

According to some embodiments, the cannabidiol containing complex has an enthalpy of about 48.3 J/g.

According to some embodiments, the cannabidiol containing complex has an enthalpy of about 19.8 J/g.

According to some embodiments, the complex of the present invention comprises (or consisting essentially of) cannabidiol and glucosamine in a molar ratio of 1:2 (cannabidiol: glucosamine), said complex having one, two, three, four or all five of the following characteristics a) to e):
a) the solubility of the cannabidiol in water is at least about 25 mg/L, such as in the range of from about 25 mg/L to about 30 mg/L, preferably from about 26 mg/L to about 27 mg/L;
b) a 1H NMR (400 MHz, DMSO-d6) spectrum with characteristic peaks of δ 8.676 ppm for protons of OH groups at positions 1′ and 5′; preferably said complex has the following 1H NMR (400 MHz, DMSO-d6) spectrum: δ 8.676 (s, 2H), 6.01 (s, 2H), 5.07 (s, 1H), 4.48 (d, 1H), 4.40 (m, 1H), 3.82 (dm, 1H), 3.02 (m, 1H), 2.29 (t, 2H), 2.10 (m, 1H), 1.91 (m, 1H), 1.66 (m, 2H), 1.59 (s, 3H), 1.57 (s, 3H), 1.46 (m, 2H) 1.26 (m, 4H), 0.85 (t, 3H);
c) the following differences in the relative intensity, frequency and/or band shape in the Attenuated Total Reflectance Fourier-Transform Infrared (ATR-FTIR) spectrum: the bands at 3400 cm⁻¹ and 3520 cm⁻¹ have a decrease in intensity of at least about 5%, such as about 10%, and the band at 2750 cm⁻¹ has an increase in intensity of at least about 5%, such as about 10%, in comparison to the physical mixture;
d) a transition temperature from about 62 to about 70° C., such as of about 66.0° C., during first heating (as measured by DSC); and
e) an enthalpy from about 40 to about 60 J/g, such as about 48.3 J/g.

According to some embodiments, the complex of the present invention comprises (or consisting essentially of) cannabidiol and glucosamine in a molar ratio of 1:1 (cannabidiol: glucosamine), said complex having one, two, three or all four of the following characteristics a) to d):
a) the solubility of the cannabidiol in water is at least about 20 mg/L, such as in the range from about 20 mg/L to about 25 mg/L, preferably from about 20 mg/L to about 22 mg/L;
b) a 1H NMR (400 MHz, DMSO-d6) spectrum with characteristic peaks of δ 8.672 ppm for protons of OH groups at positions 1′ and 5′; preferably said complex has the following 1H NMR (400 MHz, DMSO-d6) spectrum: δ 8.672 (s, 2H), 6.01 (s, 2H), 5.07 (s, 1H), 4.48 (d, 1H), 4.40 (m, 1H), 3.82 (dm, 1H), 3.02 (m, 1H), 2.29 (t, 2H), 2.10 (m, 1H), 1.91 (m, 1H), 1.66 (m, 2H), 1.59 (s, 3H), 1.57 (s, 3H), 1.46 (m, 2H) 1.26 (m, 4H), 0.85 (t, 3H);
c) a transition temperature in the range from about 62 to about 70° C., such as of about 65.8° C., during first heating (as measured by DSC); and
d) an enthalpy from about 30 to 50 J/g, such as of about 38.7 J/g.

According to some embodiments, the complex of the present invention comprises (or consisting essentially of) cannabidiol and glucosamine in a molar ratio of 1:3 (cannabidiol: glucosamine), said complex having one, two, three or all four of the following characteristics a) to d):
a) the solubility of the cannabidiol in water is at least about 25 mg/L, such as in the range from about 25 mg/L to about 30 mg/L, preferably from about 27 mg/L to about 29 mg/L;
b) a 1H NMR (400 MHz, DMSO-d6) spectrum with characteristic peaks of δ 8.676 ppm for protons of OH groups at positions 1′ and 5′; preferably said complex has the following 1H NMR (400 MHz, DMSO-d6) spectrum: δ 8.676 (s, 2H), 6.01 (s, 2H), 5.07 (s, 1H), 4.48 (d, 1H), 4.40 (m, 1H), 3.82 (dm, 1H), 3.02 (m, 1H), 2.29 (t, 2H), 2.10 (m, 1H), 1.91 (m, 1H), 1.66 (m, 2H), 1.59 (s, 3H), 1.57 (s, 3H), 1.46 (m, 2H) 1.26 (m, 4H), 0.85 (t, 3H);
c) a transition temperature from about 62 to about 70° C., such as of about 66.4° C., during first heating (as measured by DSC), and
d) an enthalpy from about 15 to about 25 J/g, such as of about 19.8 J/g.

According to some embodiments, the cannabinoid is cannabidiolic acid (CBDA).

According to some embodiments, the cannabidiolic acid containing complex is characterized in that the solubility of the cannabinoid in water is at least about 30 mg/L, such as at least about 35 mg/L, at least about 40 mg/L, at least about 45 mg/L, at least about 50 mg/L, at least about 55 mg/L, at least about 60 mg/L, at least about 65 mg/L, at least about 70 mg/L, at least about 75 mg/L, at least about 80 mg/L, at least about 85 mg/L, at least about 90 mg/L, at least about 95 mg/L, or at least about 99 mg/L.

According to some embodiments, the cannabidiolic acid containing complex is characterized in that the solubility of the cannabinoid in water is at least about 75 mg/L, such as at least about 80 mg/L, at least about 85 mg/L, at least about 90 mg/L, at least about 95 mg/L, or at least about 99 mg/L.

According to some embodiments, the cannabidiolic acid containing complex is characterized in that the solubility of the cannabinoid in water is in the range from about 30 mg/L to about 110 mg/L.

According to some embodiments, the cannabidiolic acid containing complex is characterized in that the solubility of the cannabinoid in water is in the range from about 30 mg/L to about 35 mg/L.

According to some embodiments, the cannabidiolic acid containing complex is characterized in that the solubility of the cannabinoid in water is in the range from about 32 mg/L to about 34 mg/L.

According to some embodiments, the cannabidiolic acid containing complex is characterized in that the solubility of the cannabinoid in water is in the range from about 80 mg/L to about 105 mg/L.

According to some embodiments, the cannabidiolic acid containing complex is characterized in that the solubility of the cannabinoid in water is in the range from about 80 mg/L to about 90 mg/L.

According to some embodiments, the cannabidiolic acid containing complex is characterized in that the solubility of the cannabinoid in water is in the range from about 85 mg/L to about 87 mg/L.

According to some embodiments, the cannabidiolic acid containing complex is characterized in that the solubility of the cannabinoid in water is in the range from about 100 mg/L to about 105 mg/L.

According to some embodiments, the cannabidiolic acid containing complex is characterized in that the solubility of the cannabinoid in water is in the range from about 101 mg/L to about 103 mg/L.

According to some embodiments, the cannabidiolic acid containing complex is characterized in that the stability of the cannabidiolic acid in water is increased by at least about 10%, such as at least about 20%, after heating at 60° C. for 3 days compared to the stability of the cannabinoid in the absence of glucosamine under comparable conditions.

According to some embodiments, the cannabidiolic acid containing complex has a 1H NMR (400 MHz, DMSO-d6) spectrum with a characteristic peak of from δ 15.1 to δ 16.7 ppm for proton of OH group at position 1′.

According to some embodiments, the cannabidiolic acid containing complex has a 1H NMR (400 MHz, DMSO-d6) spectrum with a characteristic peak of δ 15.1 ppm for proton of OH group at position 1′.

According to some embodiments, the cannabidiolic acid containing complex has a 1H NMR (400 MHz, DMSO-d6) spectrum with a characteristic peak of δ 16.5 ppm for proton of OH group at position 1′.

According to some embodiments, the cannabidiolic acid containing complex has a 1H NMR (400 MHz, DMSO-d6) spectrum with a characteristic peak of δ 16.7 ppm for proton of OH group at position 1′.

According to some embodiments, the cannabidiolic acid containing complex has a 1H NMR (400 MHz, DMSO-d6) spectrum with a characteristic peak of from δ 5.84 to 6.01 ppm for proton at position 4′.

According to some embodiments, the cannabidiolic acid containing complex has a 1H NMR (400 MHz, DMSO-d6) spectrum with a characteristic peak of δ 5.84 ppm for proton at position 4′.

According to some embodiments, the cannabidiolic acid containing complex has a 1H NMR (400 MHz, DMSO-d6) spectrum with a characteristic peak of δ 5.86 ppm for proton at position 4′.

According to some embodiments, the cannabidiolic acid containing complex has a 1H NMR (400 MHz, DMSO-d6) spectrum with a characteristic peak of δ 6.01 ppm for proton at position 4′.

According to some embodiments, the cannabidiolic acid containing complex has a 1H

NMR (400 MHz, DMSO-d6) spectrum with a characteristic peak of from δ 8.73 to 9.08 ppm for proton of OH group at position 5′.

According to some embodiments, the cannabidiolic acid containing complex has a 1H NMR (400 MHz, DMSO-d6) spectrum with a characteristic peak of δ 8.73 ppm for proton of OH group at position 5′.

According to some embodiments, the cannabidiolic acid containing complex has a 1H NMR (400 MHz, DMSO-d6) spectrum with a characteristic peak of δ 8.80 ppm for proton of OH group at position 5′.

According to some embodiments, the cannabidiolic acid containing complex has a 1H NMR (400 MHz, DMSO-d6) spectrum with a characteristic peak of δ 9.08 ppm for proton of OH group at position 5′.

According to some embodiments, the cannabidiolic acid containing complex has a 1H NMR spectrum wherein the signal for 2′ COOH proton is not present.

According to some embodiments, the cannabidiolic acid containing complex has a 1H NMR (400 MHz, DMSO-d6) spectrum with a characteristic peak of from δ 15.1 to δ 16.7 ppm for proton of OH group at position 1′; a characteristic peak of from δ 5.84 to 6.01 ppm for proton at position 4′; a characteristic peak of from δ 8.73 to 9.08 ppm for proton of OH group at position 5′; and the signal for 2′ COOH proton not being present.

According to some embodiments, the cannabidiolic acid containing complex has a 1H NMR (400 MHz, DMSO-d6) spectrum with a characteristic peak of δ 15.1 ppm for proton of OH group at position 1′, a characteristic peak of δ 6.01 ppm for proton at position 4′, a characteristic peak of δ 9.08 ppm for proton of OH group at position 5′, and the signal for 2′ COOH proton not being present.

According to some embodiments, the cannabidiolic acid containing complex has a 1H NMR (400 MHz, DMSO-d6) spectrum with a characteristic peak of δ 16.5 ppm for proton of OH group at position 1′, a characteristic peak of δ 5.86 ppm for proton at position 4′, a characteristic peak of δ 8.80 ppm for proton of OH group at position 5′, and the signal for 2′ COOH proton not being present.

According to some embodiments, the cannabidiolic acid containing complex has a 1H NMR (400 MHz, DMSO-d6) spectrum with a characteristic peak of δ 16.7 ppm for proton of OH group at position 1′, a characteristic peak of δ 5.84 ppm for proton at position 4′, a characteristic peak of 8.73 ppm for proton of OH group at position 5′, and and the signal for 2′ COOH proton not being present.

According to some embodiments, the cannabidiolic acid containing complex has the following 1H NMR (400 MHz, DMSO-d6) spectrum: δ 15.1 (s, 1H), 9.08 (s, 1H), 5.94 (s, 1H), 5.06 (s, 1H), 4.48 (d, 1H), 4.39 (m, 1H), 3.87 (m, 1H), 2.09 (m, 1H), 1.91 (m, 1H), 1.67 (m, 2H), 1.59 (s, 3H), 1.58 (s, 3H), 1.45 (m, 2H) 1.25 (m, 4H), 0.85 (t, 3H).

According to some embodiments, the cannabidiolic acid containing complex has the following 1H NMR (400 MHz, DMSO-d6) spectrum: δ 16.5 (s, 1H), 8.80 (s, 1H), 5.86(s, 1H), 5.05 (s, 1H), 4.49 (d, 1H), 4.38 (m, 1H), 3.86 (m, 1H), 2.09 (m, 1H), 1.90 (m, 1H), 1.66 (m, 2H), 1.58 (s, 3H), 1.58 (s, 3H), 1.44 (m, 2H) 1.25 (m, 4H), 0.85 (t, 3H).

According to some embodiments, the cannabidiolic acid containing complex has the following 1H NMR (400 MHz, DMSO-d6) spectrum: δ 16.7 (s, 1H), 8.73 (s, 1H), 5.84(s, 1H), 5.05 (s, 1H), 4.49 (d, 1H), 4.38 (m, 1H), 3.86 (m, 1H), 2.09 (m, 1H), 1.90 (m, 1H), 1.66 (m, 2H), 1.58 (s, 3H), 1.58 (s, 3H), 1.44 (m, 2H) 1.25 (m, 4H), 0.85 (t, 3H).

According to some embodiments, the cannabidiolic acid containing complex has the following differences in the relative intensity, frequency and/or band shape in the Attenuated Total Reflectance Fourier-Transform Infrared (ATR-FTIR) spectrum: the band at 3300 cm⁻¹ has a decrease in intensity of at least about 15%, such as about 25%, the band at 1372 cm⁻¹ has an increase in intensity of at least about 80%, such as about 95%, and the band at 2300 cm⁻¹ has an increase in intensity of at least about 20%, such as about 33%, in comparison to the physical mixture.

According to some embodiments, the cannabidiolic acid containing complex has a first transition temperature in the range from about 30° C. to about 100° C., such as of about 73.6° C., a second transition temperature in the range from about 100° C. to about 106° C., such as of about 104.6° C., and a third transition temperature in the range from about 106° C. to about 110° C., such as of about 107.4° C., during first heating (as measured by DSC).

According to some embodiments, the cannabidiolic acid containing complex has a first transition temperature in the range from about 60° C. to about 80° C., a second transition temperature in the range from about 103° C. to about 105° C. and a third transition temperature in the range from about 106° C. to about 109° C. during first heating (as measured by DSC).

According to some embodiments, the cannabidiolic acid containing complex has a first transition temperature in the range from about 70° C. to about 75° C., a second transition temperature in the range from about 104° C. to about 105° C., and a third transition temperature in the range from about 107° C. to about 108° C. during first heating (as measured by DSC).

According to some embodiments, the cannabidiolic acid containing complex has a first transition temperature of about 73.6° C., a second transition temperature of about 104.6° C., and a third transition temperature of about 107.4° C. during first heating (as measured by DSC).

According to some embodiments, the cannabidiolic acid containing complex has a first transition enthalpy in the range from about 10 to about 50 J/g, such as of about 38.4 J/g, a second transition enthalpy in the range from about 0.1 to about 1.0 J/g, such as of about 0.3 J/g, and a third transition enthalpy in the range from about 0.1 to about 1.0 J/g, such as of about 0.4 J/g, during first heating (as measured by DSC).

According to some embodiments, the cannabidiolic acid containing complex has a first transition enthalpy in the range from about 35 to about 40 J/g, a second transition enthalpy in the range from about 0.1 to about 0.5 J/g, and a third transition enthalpy in the range from about 0.1 to about 0.5 J/g during first heating (as measured by DSC).

According to some embodiments, the cannabidiolic acid containing complex has a first transition enthalpy in the range from about 35 to about 40 J/g, a second transition enthalpy in the range from about 0.2 to about 0.4 J/g, and a third transition enthalpy in the range from about 0.3 to about 0.5 J/g, during first heating (as measured by DSC).

According to some embodiments, the cannabidiolic acid containing complex has a first transition enthalpy of about 38.4 J/g, a second transition enthalpy of about 0.3 J/g, and a third transition enthalpy of about 0.4 J/g during first heating (as measured by DSC).

According to some embodiments, the complex of the present invention comprises (or consisting essentially of) cannabidiolic acid and glucosamine in a molar ratio of 1:2 (cannabidiolic acid: glucosamine), said complex having one, two, three, four or all five of the following characteristics a) to e):
a) the solubility of the cannabidiolic acid in water is at least about 100 mg/L, such as in the range from about 100 mg/L to about 105 mg/L, preferably from about 101 mg/L to about 103 mg/L;
b) a 1H NMR (400 MHz, DMSO-d6) spectrum with a characteristic peak of δ 16.5 ppm for proton of OH group at position 1′, a characteristic peak of δ 5.86 ppm for proton at position 4′, a characteristic peak of δ 8.80 ppm for proton of OH group at position 5′, and the signal for 2′ COOH proton not being present; preferably said complex has the following 1H NMR (400 MHz, DMSO-d6) spectrum: δ 16.5 (s, 1H), 8.80 (s, 1H), 5.86 (s, 1H), 5.05 (s, 1H), 4.49 (d, 1H), 4.38 (m, 1H), 3.86 (m, 1H), 2.09 (m, 1H), 1.90 (m, 1H), 1.66 (m, 2H), 1.58 (s, 3H), 1.58 (s, 3H), 1.44 (m, 2H) 1.25 (m, 4H), 0.85 (t, 3H);
c) following differences in the relative intensity, frequency and/or band shape in the Attenuated Total Reflectance Fourier-Transform Infrared (ATR-FTIR) spectrum: the band at 3300 cm⁻¹ has a decrease in intensity of at least about 15%, such as about 25%, the band at 1372 cm⁻¹ has an increase in intensity of at least about 80%, such as about 95%, and the band at 2300 cm⁻¹ has an increase in intensity of at least about 20%, such as about 33%, in comparison to the physical mixture;
d) a first transition temperature in the range from about 60° C. to about 80° C., such as of about 73.6° C., a second transition temperature in the range from about 103° C. to about 105° C., such as of about 104.6° C., and a third transition temperature in the range from about 106° C. to about 109° C., such as of about 107.4° C., during first heating (as measured by DSC); and
e) a first transition enthalpy in the range from about 35 to about 40 J/g, such as of about 38.4 J/g, a second transition enthalpy in the range from about 0.2 to about 0.4 J/g, such as of about 0.3 J/g, and a third transition enthalpy in the range from about 0.3 to about 0.5 J/g, such as of about 0.4 J/g, during first heating (as measured by DSC).

According to some embodiments, the cannabinoid is cannabigerol (CBG).

According to some embodiments, the cannabigerol containing complex is characterized in that the solubility of the cannabinoid in water is at least about 15 mg/L, such as at least about 20 mg/L, at least about 25 mg/L, at least about 30 mg/L, at least about 35 mg/L, at least about 40 mg/L, at least about 45 mg/L, at least about 50 mg/L, at least about 55 mg/L, at least about 60 mg/L, at least about 65 mg/L, at least about 70 mg/L, at least about 75 mg/L, at least about 80 mg/L, at least about 85 mg/L, at least about 90 mg/L, at least about 95 mg/L, or at least about 99 mg/L.

According to some embodiments, the cannabigerol containing complex is characterized in that the solubility of the cannabinoid in water is at least about 25 mg/L, such as at least about 30 mg/L, at least about 35 mg/L, at least about 40 mg/L, at least 45 mg/L, at least about 50 mg/L, at least about 55 mg/L, at least about 60 mg/L, at least about 65 mg/L, at least about 70 mg/L, at least about 75 mg/L, at least about 80 mg/L, at least about 85 mg/L, at least about 90 mg/L, at least about 95 mg/L, or at least about 99 mg/L.

According to some embodiments, the cannabigerol containing complex is characterized in that the solubility of the cannabinoid in water is in the range from about 15 mg/L to about 35 mg/L, such as from about 18 mg/L to about 32 mg/L or from about 20 mg/L to about 30 mg/L.

According to some embodiments, the cannabigerol containing complex is characterized in that the solubility of the cannabinoid in water is in the range from 18 mg/L to about 32 mg/L.

According to some embodiments, the cannabigerol containing complex is characterized in that the solubility of the cannabinoid in water is in the range from about 20 mg/L to about 30 mg/L.

According to some embodiments, the cannabigerol containing complex has a 1H NMR (400 MHz, DMSO-d6) spectrum with characteristic peaks of δ 8.89 ppm for protons of OH groups at positions 1 and 5.

According to some embodiments, the cannabigerol containing complex has the following 1H NMR (400 MHz, DMSO-d6) spectrum: δ 8.89 (s, 2H), 6.08 (s, 2H), 5.15 (m, 1H), 5.04 (m, 1H), 3.11 (d, 2H), 1.97 (m, 2H), 1.87 (m, 2H), 1.68 (s, 3H), 1.60 (s, 3H), 1,52 (s, 3H), 1.46 (m, 2H), 1.26 (m, 4H), 0.85 (t, 3H).

According to some embodiments, the cannabigerol containing complex has the following differences in the relative intensity, frequency and/or band shape in the Attenuated Total Reflectance Fourier-Transform Infrared (ATR-FTIR) spectrum: the band at 2750 cm⁻¹ has a decrease in intensity of at least about 10%, such as about 15%, and the band at 3300 cm⁻¹ has an increase in intensity of at least about 10%, such as about 15%, in comparison to the physical mixture.

According to some embodiments, the cannabigerol containing complex has a transition temperature in the range from about 40° C. to about 70° C., such as of about 53.1° C., during first heating (as measured by DSC).

According to some embodiments, the cannabigerol containing complex has a transition temperature in the range from about 50° C. to about 60° C. during first heating (as measured by DSC).

According to some embodiments, the cannabigerol containing complex has a transition temperature in the range from about 50° C. to about 55° C. during first heating (as measured by DSC).

According to some embodiments, the cannabigerol containing complex has a transition temperature of about 53.1° C., during first heating (as measured by DSC).

According to some embodiments, the cannabigerol containing complex has a transition enthalpy in the range from about 30 to about 70 J/g, such as of about 51.2 J/g, during first heating (as measured by DSC).

According to some embodiments, the cannabigerol containing complex has a transition enthalpy in the range from about 40 to about 60 J/g during first heating (as measured by DSC).

According to some embodiments, the cannabigerol containing complex has a transition enthalpy in the range from about 45 to about 55 J/g during first heating (as measured by DSC).

According to some embodiments, the cannabigerol containing complex has a transition enthalpy of about 51.2 J/g during first heating (as measured by DSC).

According to some embodiments, the complex of the present invention comprises (or consisting essentially of) cannabigerol and glucosamine in a molar ratio of 1:2 (cannabigerol: glucosamine), said complex having one, two, three, four or all five of the following characteristics a) to e):
a) the solubility of the cannabigerol in water is at least about 25 mg/L, such as in the range from about 28 mg/L to about 32 mg/L, preferably from about 29 mg/L to about 30 mg/L;
b) a 1H NMR (400 MHz, DMSO-d6) spectrum with characteristic peaks of δ 8.89 ppm for protons of OH groups at positions 1 and 5; preferably said complex has the following 1H NMR (400 MHz, DMSO-d6) spectrum: δ 8.89 (s, 2H), 6.08 (s, 2H), 5.15 (m, 1H), 5.04 (m, 1H), 3.11 (d, 2H), 1.97 (m, 2H), 1.87 (m, 2H), 1.68 (s, 3H), 1.60 (s, 3H), 1.52 (s, 3H), 1.46 (m, 2H), 1.26 (m, 4H), 0.85 (t, 3H);
c) the following differences in the relative intensity, frequency and/or band shape in the Attenuated Total Reflectance Fourier-Transform Infrared (ATR-FTIR) spectrum: the band at 2750 cm⁻¹ has a decrease in intensity of at least about 10%, such as about 15%, and the band at 3300 cm⁻¹ has an increase in intensity of at least about 10%, such as about 15%, in comparison to the physical mixture;
d) a transition temperature in the range from about 50° C. to about 55° C., such as of about 53.1° C., during first heating (as measured by DSC); and
e) a transition enthalpy in the range from about 45 to about 55 J/g, such as of about 51.2 J/g, during first heating (as measured by DSC).

The transition temperature and enthalpy can be determined using the following DSC measurement: Differential-scanning calorimetry (DSC) measurements are done on a Perkin Elmer (Waltham, MA, USA) Pyris 1 instrument. In the sample cup, 1.5 to 6 mg of the sample is weighted. Measurements are done in the temperature range between 25 and 110° C. in an inert nitrogen atmosphere (nitrogen flow 20 ml/min), consisting of a first heating at 10° C./min, then cooling afterwards at −1° C./min and a second heating step (also at 10° C./min). Changes in sample heat exchange (enthalpy) are recorded. For characterisation purposes, only measurements during the first heating step are used.

Attenuated Total Reflectance Fourier-Transform Infrared (ATR-FTIR) spectrum can be determined by the following ATR-FTIR measurement: Measurements of ATR-FTIR spectra are performed at 298 K on the Bruker (Billerica, MA, USA) Tensor 27 spectrometer using a single reflectance Specac (Orpington, Kent, UK) Golden Gate diamond ATR accessory. A nitrogen-cooled MCT detector is used, and the spectrometer optics and ATR cell are sealed against the atmosphere and purged with technical dry nitrogen during measurements. ATR-FTIR spectra are recorded in the range from 4000 cm⁻¹ to 600 cm⁻¹. The spectrum is obtained by averaging 128 scans with a nominal resolution of 2 cm⁻¹. The temperature is controlled using a Specac Heated Golden Gate Controller. Prior to the application of each sample and measurement, the surface of the diamond ATR crystal is cleaned with acetone. Water and CO2 atmospheric compensation is applied to the obtained spectra.

NMR spectrum may be determined by the following measurement: Liquid-state NMR experiments are performed on a 400 MHz Bruker (Billerica, MA, USA) AVANCE NEO NMR spectrometer using a 5 mm BB(F)O Iprobe. Two to seven milligrams of a sample are dissolved in 600 μL of DMSO-d6. 1H NMR spectra are then acquired using zg pulse sequence at 25° C. A total of 128 scans are accumulated with the repetition delay of 3 s, with a spectral width of 8.6 kHz. The chemical shifts are referenced on signal of DMSO. 1H-13C HSQC spectra are in addition used for assignment of individual signals.

According to some embodiments, the complex of the present invention is obtainable by a process for preparation as detailed below.

The present invention provides in a further aspect a composition comprising a) a cannabinoid and b) glucosamine. The cannabinoid component may be one as detailed above.

According to some embodiments, the composition is characterized in that the solubility of the cannabinoid in water is at least about 15 mg/L, such as at least about 20 mg/L, at least about 25 mg/L, at least about 30 mg/L, at least about 35 mg/L, at least about 40 mg/L, at least about 45 mg/L, at least about 50 mg/L, at least about 55 mg/L, at least about 60 mg/L, at least about 65 mg/L, at least about 70 mg/L, at least about 75 mg/L, at least about 80 mg/L, at least about 85 mg/L, at least about 90 mg/L, at least about 95 mg/L, or at least about 99 mg/L.

According to some embodiments, the composition is characterized in that the solubility of the cannabinoid in water is at least about 25 mg/L, such as at least about 30 mg/L, at least about 35 mg/L, at least about 40 mg/L, at least about 45 mg/L, at least about 50 mg/L, at least about 55 mg/L, at least about 60 mg/L, at least about 65 mg/L, at least about 70 mg/L, at least about 75 mg/L, at least about 80 mg/L, at least about 85 mg/L, at least about 90 mg/L, at least about 95 mg/L, or at least about 99 mg/L.

According to some embodiments, the composition is characterized in that the solubility of the cannabinoid in water is at least about 30 mg/L, such as at least about 35 mg/L, at least about 40 mg/L, at least about 45 mg/L, at least about 50 mg/L, at least about 55 mg/L, at least about 60 mg/L, at least about 65 mg/L, at least about 70 mg/L, at least about 75 mg/L, at least about 80 mg/L, at least about 85 mg/L, at least about 90 mg/L, at least about 95 mg/L, or at least about 99 mg/L.

According to some embodiments, the composition is characterized in that the solubility of the cannabinoid in water is at least about 50 mg/L, such as at least about 55 mg/L, at least about 60 mg/L, at least about 65 mg/L, at least about 70 mg/L, at least about 75 mg/L, at least about 80 mg/L, at least about 85 mg/L, at least about 90 mg/L, at least about 95 mg/L, or at least about 99 mg/L.

According to some embodiments, the composition is characterized in that the solubility of the cannabinoid in water is at least about at least about 75 mg/L, such as at least about 80 mg/L, at least about 90 mg/L, at least about 95 mg/L, or at least about 99 mg/L.

According to some embodiments, the composition is characterized in that the solubility of the cannabinoid in water is in the range from about 15 mg/L to about 110 mg/L, such as from about 15 mg/L to about 55 mg/L, from about 15 mg/L to about 40 mg/L from about 15 mg/L to about 35 mg/L, from about 15 mg/L to about 30 mg/L, from about 20 mg/L to about 55 mg/L, from about 20 mg/L to about 40 mg/L, from about 20 mg/L to about 35 mg/L, from about 20 mg/L to about 30 mg/L, from about 30 mg/L to about 40 mg/L, from about 30 mg/L to about 35 mg/L, from about 80 mg/L to about 110 mg/L, from about 100 mg/L to about 110 mg/L, from about 85 mg/L to about 105 mg/L or from about 80 mg/L to about 90 mg/L.

According to some embodiments, the composition is characterized in that the solubility of the cannabinoid in water is in the range from about 15 mg/L to about 35 mg/L, such as from about 15 mg/L to about 30 mg/L, from about 20 mg/L to about 35 mg/L, from about 20 mg/L to about 30 mg/L, or from about 30 mg/L to about 35 mg/L.

According to some embodiments, the composition is characterized in that the solubility of the cannabinoid in water is in the range from about 15 mg/L to about 30 mg/L.

According to some embodiments, the composition is characterized in that the solubility of the cannabinoid in water is in the range from about 15 mg/L to about 20 mg/L.

According to some embodiments, the composition is characterized in that the solubility of the cannabinoid in water is in the range from about 18 mg/L to about 22 mg/L.

According to some embodiments, the composition is characterized in that the solubility of the cannabinoid in water is in the range about 20 mg/L to about 35 mg/L.

According to some embodiments, the composition is characterized in that the solubility of the cannabinoid in water is in the range about 20 mg/L to about 30 mg/L.

According to some embodiments, the composition is characterized in that the solubility of the cannabinoid in water is in the range about 20 mg/L to about 25 mg/L. According to some embodiments, the composition is characterized in that the solubility of the cannabinoid in water is in the range about 25 mg/L to about 30 mg/L.

According to some embodiments, the composition is characterized in that the solubility of the cannabinoid in water is in the range from about 80 mg/L to about 110 mg/L.

According to some embodiments, the composition is characterized in that the solubility of the cannabinoid in water is in the range from about 85 mg/L to about 105 mg/L.

According to some embodiments, the composition is characterized in that the solubility of the cannabinoid in water is in the range from about 80 mg/L to about 90 mg/L.

According to some embodiments, the composition is characterized in that the solubility of the cannabinoid in water is in the range from about 100 mg/L to about 105 mg/L.

According to some embodiments, the composition comprises at least one complex according to the present invention.

According to some embodiments, the composition comprises a hemp extract.

The present invention provides in a further aspect a process for the preparation of a complex according to the present invention. More specifically, the process of the present invention comprises the step of reacting the cannabinoid with glucosamine base.

Suitably, the reaction between the cannabinoid and glucosamine base is carried out in one or more suitable solvents which dissolve(s) both compounds (e.g. an alcohol such as methanol or ethanol) or a mixture of solvents (e.g., 50% ethanol).

Generally, a determined molar quantity of cannabinoid is dissolved in an appropriate solvent, most conveniently in an organic solvent such as an alcohol like methanol or ethanol, or an alcoholic solution contain a certain % of water (e.g. 50% ethanol). Yet, other water-miscible solvents may also be used instead. A determined molar quantity of glucosamine base, such as freshly prepared glucosamine base (i.e. just after removal of its counter anion), in a certain molar ratio to the cannabinoid (such as a 2-fold molar quantity) is then added stepwise in an appropriate solvent. The solvent for glucosamine can be the same as for the cannabinoid or can be water.

The resulting mixture solution obtained by mixing (such as at room temperature or higher temperature) can then be evaporated (such as by use of a rotary evaporator). Eventually, if water is also present, the mixture solution can also be freeze dried afterwards.

In the solution and by removing the solvent(s), the cannabinoid and glucosamine base interact with each other, thus forming some, albeit weak, intermolecular interactions (such as Van der Waals forces), which eventually results in the formation of the complex.

Thus, according to some embodiments, the step of reacting the cannabinoid with glucosamine base comprises dissolving a determined molar quantity of the cannabinoid in an appropriate solvent, preferably an organic solvent such as an alcohol like methanol or ethanol, or an alcoholic solution contain a certain % of water (e.g. 50% ethanol); dissolving a determined molar quantity of glucosamine base to obtain a desired molar ratio to the cannabinoid (such as a 2-fold molar quantity) in an appropriate solvent, and mixing both solutions to obtain a mixture solution.

According to some embodiments, the solvent for the cannabinoid is an organic solvent, preferably an organic solvent which is volatile.

According to some embodiments, the solvent for the cannabinoid is an organic solvent selected from the group consisting of alcohol, such as methanol or ethanol, or an alcoholic solution, acetonitrile, acetone, 1-propanol, 2-propanol, chloroform etc.

According to some embodiments, the solvent for the cannabinoid is an alcohol, such as methanol or ethanol, or an alcoholic solution.

According to some embodiments, the solvent for the cannabinoid is an alcoholic solution, such as a aqueous solution containing 50% methanol or ethanol.

According to some embodiments, the solvent for glucosamine base is the same as for the cannabinoid.

According to some embodiments, the step of reacting the cannabinoid with glucosamine base further comprises evaporating the obtained mixture solution (such as by use of a rotary evaporator).

According to some embodiments, the step of reacting the cannabinoid with glucosamine base is carried out at a temperature ranging from about 10° C. to about 60° C., preferably a temperature ranging from about 20° C. to about 30° C., such as about 25° C.

According to some embodiments, the step of reacting the cannabinoid with glucosamine base is carried out for a time period from about 2 minutes to about 2 hours.

According to some embodiments, the process comprises a pre-treatment step which comprises stripping off the counter ion (i.e. anion, usually sulfate or chloride) from glucosamine in salt form (such as glucosamine hydrochloride) in order to obtain said glucosamine base.

According to some embodiments, the stripping off is facilitated by use of an appropriate means, such as by anion-exchange chromatography.

According to some embodiments, the stripping off is facilitated by anion-exchange chromatography, precipitation of the anion (e.g. by the addition of appropriate base), dialysis or reverse osmosis using an ion-selective membrane.

According to some embodiments, the pre-treatment step further comprises drying the obtained glucosamine base (e.g., by evaporation or lyophilisation or spray drying).

The present invention further provides a method for improving the solubility of a cannabinoid in water, comprising the step of reacting the cannabinoid with glucosamine base.

According to some embodiments, the step of reacting the cannabinoid with glucosamine base comprises dissolving a determined molar quantity of the cannabinoid in an appropriate solvent, preferably an organic solvent such as an alcohol like methanol or ethanol, or an alcoholic solution contain a certain % of water (e.g. 50% ethanol); dissolving a determined molar quantity of glucosamine base to obtain a desired molar ratio to the cannabinoid (such as a 2-fold molar quantity) in an appropriate solvent, and mixing both solutions to obtain a mixture solution.

According to some embodiments, the solvent for the cannabinoid is an alcohol, such as methanol or ethanol, or an alcoholic solution.

According to some embodiments, the solvent for the cannabinoid is an alcoholic solution, such as a aqueous solution containing 50% methanol or ethanol.

According to some embodiments, the solvent for glucosamine base is the same as for the cannabinoid.

According to some embodiments, the step of reacting the cannabinoid with glucosamine base further comprises evaporating the obtained mixture solution (such as by use of a rotary evaporator).

According to some embodiments, the step of reacting the cannabinoid with glucosamine base is carried out at a temperature ranging from about 10° C. to about 60° C.

According to some embodiments, the step of reacting the cannabinoid with glucosamine base is carried out for a time period from about 2 minutes to about 2 hours.

According to some embodiments, the method comprises a pre-treatment step which comprises stripping off the counter ion (i.e. anion, usually sulfate or chloride) from glucosamine in salt form (such as glucosamine hydrochloride or glucosamine sulfate) in order to obtain said glucosamine base.

According to some embodiments, the stripping off is facilitated by use of an appropriate means, such as by anion-exchange chromatography.

According to some embodiments, the pre-treatment step further comprises drying the obtained glucosamine base (e.g., by evaporation or lyophilisation).

The cannabinoid may be as defined above.

According to some embodiments, the cannabinoid is selected from the group consisting of: cannabinol (CBN), cannabinolic acid (CBNA), cannabidiol (CBD), cannabidiolic acid (CBDA), cannabigerol (CBG), cannabigerolic acid (CBGA), tetrahydrocannabinol (THC), tetrahydrocannabinolic acid (THCA), Δ(9)-tetrahydrocannabinol (D9THC), Δ(9)-tetrahydrocannabinolic acid (D9THCA), Δ(9)-cannabidiol (D9CBD), Δ(9)-tetrahydrocannabidiolic acid (D9THCA), Δ(8)-tetrahydrocannabinol (D8THC), Δ(8)-tetrahydrocannabinolic acid (D8THCA), tetrahydrocannabivarin (THCV), tetrahydrocannabivarin (THCV), tetrahydrocannabivarinic acid (THCVA), cannabichromene (CBC), cannabichromenic acid (CBCA), cannabicyclol (CBL), cannabicyclolic acid (CBLA), cannabielsoin (CBE), cannabielsoic acid (CBEA), cannabitriol (CBT), cannabitriolic acid (CBTA), Nabilone, and combinations thereof.

According to some embodiments, the cannabinoid is selected from the group consisting of cannabinol, cannabinolic acid, cannabidiol (CBD), cannabidiolic acid (CBDA), cannabigerol, cannabigerolic acid, and combinations thereof.

According to some embodiments, the cannabinoid is selected from the group consisting of cannabidiol (CBD), cannabidiolic acid (CBDA), cannabigerol (CBG), cannabigerolic acid (CBGA), and combinations thereof.

According to some embodiments, the cannabinoid is selected from the group consisting of cannabidiol (CBD), cannabidiolic acid (CBDA), and a combination thereof.

According to some embodiments, the cannabinoid is cannabidiol (CBD).

According to some embodiments, the cannabinoid is cannabidiolic acid (CBDA).

According to some embodiments, the cannabinoid is selected from the group consisting of cannabigerol (CBG), cannabigerolic acid (CBGA) and a combination thereof.

According to some embodiments, the cannabinoid is cannabigerol (CBG).

According to some embodiments, the cannabinoid is cannabigerolic acid (CBGA).

The present invention further provides the use of glucosamine base for improving the solubility and/or stability of a cannabinoid in water.

### Certain Definitions

A “complex” as used herein is meant a molecular entity formed by loose association involving a given cannabinoid and glucosamine (GA). The bonding between the two components is normally weaker than in a covalent bond, and may even be weaker than hydrogen bonds, such as on the level of Van der Waals forces. The intermolecular interactions can be observed by means of different measurements like DSC, NMR and

IR spectroscopy.

A “complex” of the present invention essentially differs from its “mixture” conterpart by having a significantly larger water solubility (for example, by a factor of at least 5-fold). Moreover, when DSC measurements are performed, the transition temperature observed during the melting is generally lower in the complex (compared to mixture), but more importantly, the observed endothermic melting process in the complex exhibits a larger enthalpy (expressed like in J/g). The melting peak(s) of the complex is also wider compared to a mere physical mixture. The latter could indicate that on the molecular level the structure in the complex is less ordered, or might have more »degrees of freedom«.

In NMR measurments some characteristic changes in the chemical shifts are generally observed in the »complexes«. By way of example, in a CBDA/glucosamine complex, the signal for COOH proton is absent due to deprotonation, and the values of chemical shifts for the OH protons are significantly higher, which implies the interactions of —OH groups with GA. In complexes of cannabinoid acidic forms (e.g. CBDA, CBGA) where the interactions are stronger, some changes in chemical shifts for other protons (aromatic core of the cannabinoid) are present as well.

The ATR-FTIR spectra also give an insight in the differences between a »complex« and »mixture«. In the »complex«, there is generally a less pronounced »fine structure« (i.e. the spectra are more »smooth«, with less sharp bands) in the spectral region approximately between 1500/cm and 500/cm compared to »mixture«. This is the spectral region where mainly ring deformations, C—H deformations, C—OH stretchings are typically observed.

As used herein, the indefinite articles “a” and “an” mean “at least one” or “one or more” unless the context clearly dictates otherwise.

As used herein, the terms “comprising”, “including”, “having” and grammatical variants thereof are to be taken as specifying the stated features, steps, or components but do not preclude the addition of one or more additional features, steps, components or groups thereof. The use of “comprising” and “comprises” as used herein is to be understood as also disclosing “consisting essentially of” and “consists essentially of” as well as “consisting of” and “consists of”, respectively.

As used herein, the term “consisting essentially of” (and grammatical variants thereof) generally means that additional materials, features, components, elements or steps may be included that do not materially affect the basic and novel characteristic(s) of the claimed invention. For example, when used in the context of the complex of the invention, the term means that the complex may contain additional features, components or elements in addition to those literally disclosed provided that these additional features, components or elements do not materially affect the basic and novel characteristic(s) of the claimed complex. For example, the complex may include additional non-essential elements such as water (e.g., in form of moisture) and/or impurities.

As used herein, the term “about” means plus or minus 5% of the numerical value of the number with which it is being used.

Where a numerical limit or range is stated herein, the endpoints are included. Also, all values and sub ranges within a numerical limit or range are specifically included as if explicitly written out.

Having generally described this invention, a further understanding can be obtained by reference to certain specific examples, which are provided herein for purposes of illustration only, and are not intended to be limiting unless otherwise specified.

### EXAMPLES

### General Methodology

How the complexe(s) is (are) prepared:

A determined molar quantity of cannabinoid (such as CBD, CBDA or CBG) is dissolved in an appropriate volume of solvent (most conveniently in an alcohol like methanol or ethanol, but other water-miscible solvents cannot be excluded; likewise, an alcoholic solution of cannabinoid may also contain a certain % of water). A determined molar quantity of freshly prepared glucosamine base (i.e. just after removal of its counter anion) in a certain molar ratio to the cannabinoid (most likely a 2-fold molar quantity) is then added stepwise in an appropriate solvent. The solvent for glucosamine can be the same as for the cannabinoid or even can be water. The resulting mixture solution obtained by mixing (most likely at room temp., but it is possible to do that at higher temps.) is then evaporated (most likely on a rotary evaporator). Eventually, if water is also present, the mixture solution can also be freeze dried afterwards. In the solution and by removing the solvent, the two molecules interact with each other, thus forming some (albeit rather weak) intermolecular interactions which we defined as “complex”. Since we detected notable differences between a simple physical mixture and the complex in all 3 cases (for CBD, CBDA and CBG) using NMR, FTIR and DSC, we thus state that the complex is therefore different from a simple physical mixture. These intermolecular forces clearly exist, but are rather weak, not in the range of a hydrogen bond, but more probably on the level of Van der Waals forces.

### Formulation of Cannabinoids and Their Acidic Forms with Increased Stability and Water Solubility

### Procedure Description

1. Industrial hemp plant material was extracted with ethanol by sonication at room temperature for 30 minutes. The extract was filtered and the solvent was evaporated under reduced pressure. As a product a hemp extract—resin was obtained.

2. Glucosamine in base form was obtained by percolating solution of glucosamine hydrochloride in water through a strong anion exchange resin (Ambersep 900, —OH form, Supelco, Bellefonte, PA, USA). A free gravity percolating anion exchange column consisted of a resin bed of about 50 mL. The resin bed was first eluted with at least 10 bed volumes (i.e. 500 mL) of distilled water. Then, 50 ml of 10% (m/V) glucosamine salt solution are passed through the column in a dropwise fashion. The eluate of glucosamine base was collected, frozen at −80° C. (or with liquid nitrogen) and freeze-dried in order to eliminate water. Purity of the product was evaluated using HPLC analysis in HILIC mode with a Thermo Sycronis HILIC column (50×2.1 mm, 1.7 μm), according to the suggested methodology published by Dolci et al., 2014.

3. Cannabidiol (>99%, isolate) was obtained by Hempika (Nova Gorica, Slovenija) and cannabigerol (99.9%) was obtained from THC Pharm (Frankfurt, Germany). Cannabidiolic acid was synthesized according to published procedure (Mechoulam, 1969) and purified by preparative “flash” chromatography. Purity of the product was evaluated by HPLC and NMR analysis.

### Preparation of Samples

Physical mixtures of compounds in a predetermined molar ratio (e.g., 1:2) were made by finely homogenising the pre-weighted compounds (e.g., 31.4 mg of CBD with 35.8 mg of glucosamine) in solid (crystalline) state in a mortar. After several (about 3) minutes of thorough mixing and pulverising the mixture with the pestle, a homogeneous mass was obtained. The so-obtained sample of a physical mixture was stored in an air-tight container, away from any source of moisture for further use.

Complexes of compounds in a predetermined molar ratio (e.g., 1:1, 1:2 and 1:3) were made by dissolving predefined quantities of each compound in an appropriate volume of an appropriate solvent. For example, 31.4 mg of CBD were dissolved in 4 mL of methanol. Likewise, 35.8 mg of glucosamine were also dissolved in 4 mL of methanol. The glucosamine solution was then slowly added (dropwise) to the CBD solution under stirring (on a magnetic stirrer) at room temperature conditions (about 25° C.) within several minutes (about 2-15 minutes). The obtained homogeneous reaction solution was then evaporated to dryness under reduced pressure at about 50° C. on a rotary evaporator (Büchi, Flawil, Switzerland). The so-obtained solid sample of a complex was stored in an air-tight container, away from any source of moisture for further use. The obtained product was applied for water solubility studies, NMR, FTIR and DSC analyses.

Alternatively, a complex was prepared from hemp resin (e.g. with a total CBD content of about 30%) with a predetermined molar ratio with glucosamine (in relation to e.g. CBD). For example, about 10 g of such hemp resin was dissolved in an appropriate volume of an appropriate solvent (e.g. about 100 mL of methanol or ethanol). Another solution was then prepared by dissolving about 3.5 g of glucosamine in an appropriate volume of an appropriate solvent (e.g. about 100 mL of methanol or ethanol). The two solutions were then combined in a similar way as complexes of pure compounds, by a slow addition of glucosamine solution to the hemp resin solution under stirring at room temperature conditions (about 25° C.) within several minutes (about 2-15 minutes). The obtained homogeneous reaction solution was then evaporated to dryness under reduced pressure at about 50° C. on a rotary evaporator. The so-obtained hemp resin with improved properties was stored in a moisture-proof container. The obtained product was applied for water solubility studies and NMR analysis.

### Analysis

### 1. Solubility Tests

For water solubility studies, 1-1.5 mg of the prepared complex was mixed with 1 mL of distilled water and stirred for 5-10 min, if necessary also sonication was applied. Afterwards the solution was centrifuged at 3.000 g for 3 minutes and the supernatant was transferred to the vials. Concentration of dissolved cannabinoids was determined by HPLC-DAD system (Agilent 1260 Infinity, Santa Clara, CA, USA).

TABLE 1
Data on complex solubility

| Complex in ratio | Water solubility of cannabinoid [mg/L] | | |
|---|---|---|---|
| (cannabinoid: GA) | CBD/GA | CBDA/GA | CBG/GA |
| 1:1 | >15 | >30 | >15 |
| 1:2 | >25 | >75 | >25 |
| 1:3 | >25 | >75 | >25 |
| pure cannabinoid | <1 | <2 | <1 |

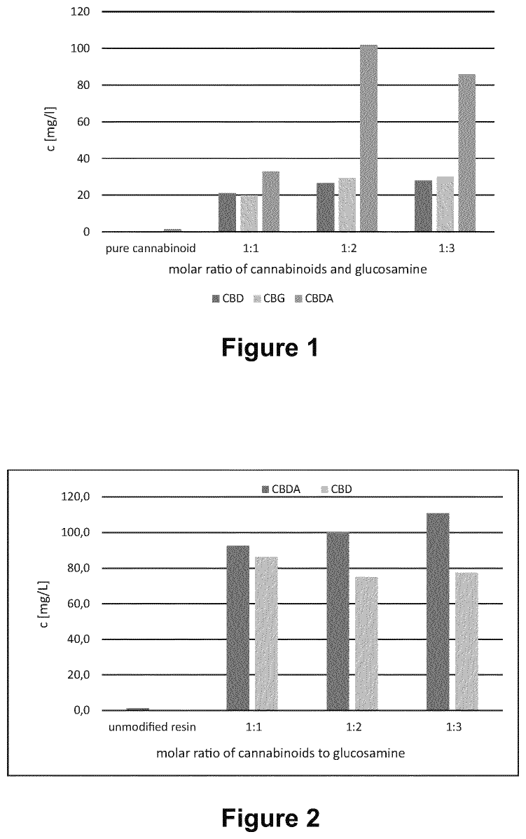

Values in the table are listed as “greater-than” or “less-than”, since the solubilities may slightly vary among experiments due to different factors (batch of prepared glucosamine and CBDA, exact mass of the complex used for solubility test, etc.). However, complexation of tested cannabinoids (CBD, CBG, CBDA) with glucosamine significantly increases their solubility in water (FIG. 1). It was discovered that the molar ratio of the cannabinoid and glucosamine in the complex considerably affects the final solubility of cannabinoid in water. For all three tested cannabinoids, the same trend regarding this aspect appeared: the cannabinoid water solubility is improved already in the complex with glucosamine in ratio 1:1, and even more in the complex 1:2. On the other hand, higher content of glucosamine in the complex (e.g. 1:3) does not improve the solubility significantly (compared to the complex 1:2).

Since it was very likely that the complex of cannabinoid and glucosamine is formed in the hemp resins (with cannabinoid content about 30%) as well, water solubility of cannabinoids was evaluated for the glucosamine modified resins. For the test, hemp resin with molar ratio of CBD:CBDA approximately 1:1 was used. In the tested concentration range (1 mg of modified hemp resin per 1 ml of water), the majority of cannabinoids was solubilised in water (>80%). Besides the mentioned resin, also a completely decarboxylated resin (only CBD) was tested; increased solubility of CBD was observed as well with the addition of glucosamine. We presume, that many factors can influence the final solubility; e.g. composition of the specific resin (other components, potential cosolvatation), as well as dynamics of adding of glucosamine to the starting material. These are probably also the reasons for significantly higer values of dissolved CBD, compared to the pure CBD-GA complexes.

TABLE 2
Concentration of cannabinoids in water, when dissolving unmodified resin
and glucosamine modified resin in the ratio cannabinoids:glucosamine 1:2.

| Hemp resin sample | CBDA conc. (mg/L) | CBD conc. (mg/L) |
|---|---|---|
| unmodified | 1.1 | n.d. |
| modified with glucosamine | 99.7 | 75.2 |

(1:2)

The effect of complex molar ratio (cannabinoids:GA) on final cannabinoid solubility was evaluated also for hemp resins (FIG. 2). Amount of added glucosamine was calculated due to the previously determined content of total cannabinoids in the resin. Same, but less evident trend was observed in the case of resins. This might be the consequence of the hemp resin composition complexity.

### 2. Stability Tests

Since the mechanism of CBDA solubilisation with glucosamine is probably connected with molecular (or ionic) interactions between carboxylic group of CBDA and amino group of glucosamine, we assumed that the potential resulting adduct could also be more stable in the aspect of CBDA decarboxylation. Since the dynamics of the decarboxylation is highly dependent on the conditions (among others, also the concentration of CBDA), we tried to provide comparable conditions for stability tests.

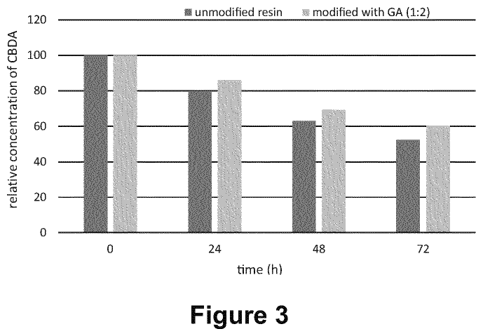

Hemp plant material with higher CBDA content was extracted in water under sonication for 30 minutes (at room temperature, without any additives). In the obtained solution, the concentration of CBDA was 13.5 mg/L. Two aliquots of the obtained solution were used for tests: to one no glucosamine was added and to the other glucosamine in molar ratio (cannabinoid: glucosamine) 1:2 was added. Both solutions were heated for 3 days at 60° C. Solutions were sampled and analysed throughout the period. In the solution with added glucosamine ca. 20% more CBDA was present after 3 days of heating (FIG. 3).

Comment regarding CBDA solubility: when prepared as a direct plant extract, the obtained CDBA concentration in water is higher compared to pure CBDA compound. This is most likely due to the presence of various compounds from hemp plant material which enhance the co-solubilisation of CBDA.

### 3. NMR Analysis

NMR analysis of starting compounds (CBDA, CBD, glucosamine) as well as the preformed complexes (CBD-glucosamine, CBDA-glucosamine) has been performed in solution of aprotic solvent (DMSO-d6). Observable and in some case significant shifts in the spectra have been found. The data below under points 3.1, 3.2, 3.3 are for 1:2 complex.

Liquid-state NMR experiments were performed on a Bruker AVANCE NEO 400 MHz NMR spectrometer using a 5 mm BB(F)O Iprobe. 2-7 mg of samples were dissolved in 600 μL of DMSO-d6. 1H NMR spectra were acquired using zg pulse sequence at 25° C. A total of 128 scans were accumulated with the repetition delay of 3 s. Spectral width was 8.6 kHz. The chemical shifts were referenced on signal of DMSO. ¹H-¹³C HSQC spectra were in addition used for assignment of individual signals.

### 3.1 CBD

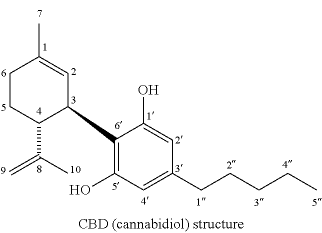 [SMILES: C=C(C)[C@@H]1CCC(C)=C[C@H]1c1c(O)cc(CCCCC)cc1O]

TABLE 3
NMR data for CBD and CBD-glucosamine complex

| | multiplicity | δ | multiplicity (GA | δ (GA |
|---|---|---|---|---|
| H | (pure) | (pure) | complex) | complex) |
| 2 (1H) | s | 5.07 | s | 5.07* |
| 3 (1H) | dm | 3.82 | dm | 3.82* |
| 4 (1H) | m | 3.02 | m | 3.02* |
| 5 (2H) | m | 1.66 | m | 1.66 |
| 6 (1H) | m | 1.91 | m | 1.91 |
| 6 (1H) | m | 2.10 | m | 2.10* |
| 7 (3H) | s | 1.59 | s | 1.59 |
| 9 (1H) | m | 4.40 | m | 4.40 |
| 9 (1H) | d | 4.48 | d | 4.48 |
| 10 (3H) | s | 1.57 | s | 1.57 |
| 2′,4′(2H) | s | 6.01 | s | 6.01 |
| 1′,5′OH | s | 8.669 | s (broad signal) | 8.676 |
| (2H) | | | | |
| 1″ (2H) | t | 2.29 | t | 2.29 |
| 2″ (2H) | m | 1.46 | m | 1.46 |
| 3″,4″ (4H) | m | 1.26 | m | 1.26 |
| 5″ (3H) | t | 0.85 | t | 0.85 |

*peak is overlapped with peak of glucosamine protons

Compared to the pure CBD, in the complex with GA a slight change in the chemical shift occurs (from δ 8.669 ppm to δ 8.676 ppm) for protons of OH groups at positions 1′ and 5′. However, a noticeable change in the peak shape is observed as well; the mentioned peak in complex with GA is broader. This indicates a weak interaction with present glucosamine.

TABLE 4
Characteristic NMR data for CBD-GA complex
(compared to the pure CBD)

| | δ (GA complex) | δ (pure) | Difference in peak |
|---|---|---|---|
| H | [ppm] | [ppm] | shape |
| OH protons | 8.676 | 8.669 | peak is broader in the |
| (positions 1′ and | | | CBD-GA complex |
| 5′) | | | |

### 3.2 CBG

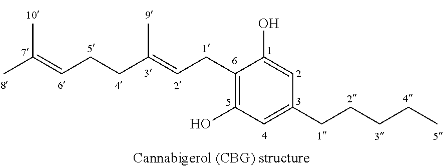 [SMILES: CCCCCc1cc(O)c(C/C=C(\C)CCC=C(C)C)c(O)c1]

TABLE 5
NMR data for CBG and CBG-glucosamine complex

| | multiplicity | | mutlitplicity (GA | δ (GA |
|---|---|---|---|---|
| H | (pure) | δ (pure) | complex) | complex) |
| 2,4 (2H) | s | 6.08 | s | 6.08 |
| 1,5 OH | s | 8.88 | s (broad signal) | 8.89 |
| (2H) | | | | |
| 1′ (2H) | d | 3.11 | d | 3.11 |
| 2′ (1H) | m | 5.15 | m | 5.15* |
| 4′ (2H) | m | 1.87 | m | 1.87 |
| 5′(2H) | m | 1.97 | m | 1.97* |
| 6′ (1H) | m | 5.04 | m | 5.04* |
| 8′ (3H) | s | 1.60 | s | 1.60 |
| 9′ (3H) | s | 1.68 | s | 1.68 |
| 10′ (3H) | s | 1.52 | s | 1.52 |
| 1″ (2H) | t | 2.31 | | n.a. |
| 2″ (2H) | m | 1.46 | m | 1.46 |
| 3″,4″ (4H) | m | 1.26 | m | 1.26 |
| 5″ (3H) | t | 0.85 | t | 0.85 |

n.a. . . . not assigned
*peak is overlapped with peak of glucosamine protons

Compared to the pure CBG, in the complex with GA a slight change in the chemical shift occurs (from δ 8.88 ppm to δ 8.89 ppm) for protons of OH groups at positions 1 and 5. However, a noticeable change in the peak shape is observed as well; the mentioned peak in complex with GA is broader. This indicates a weak interaction with present glucosamine.

TABLE 6
Characteristic NMR data for CBG-GA complex (compared to the pure CBG)

| | δ (GA complex) | δ (pure) | Difference in peak |
|---|---|---|---|
| H | [ppm] | [ppm] | shape |
| OH protons | 8.89 | 8.88 | peak is broader in the |
| (positions 1 and | | | CBG-GA complex |
| 5) | | | |

### 3.3 CBDA

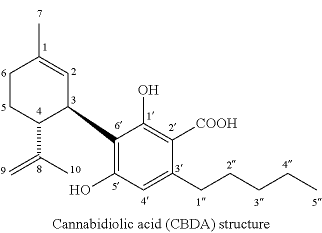 [SMILES: C=C(C)[C@@H]1CCC(C)=C[C@H]1c1c(O)cc(CCCCC)c(C(=O)O)c1O]

TABLE 7
NMR data for CBDA and CBDA-glucosamine complex

| | multiplicity | δ (pure) | multiplicity (GA | δ (GA complex) |
|---|---|---|---|---|
| H | (pure) | [ppm] | mod.) | [ppm] |
| 2 (1H) | s | 5.07 | s | 5.05 |
| 3 (1H) | dm | 3.88 | m | 3.86 |
| 4 (1H) | m | 3.03 | | n.a. |
| 5 (2H) | m | 1.67 | m | 1.66 |
| 6 (1H) | m | 1.93 | m | 1.90 |
| 6 (1H) | m | 2.10 | m | 2.09 |
| 7 (3H) | s | 1.60 | s | 1.58* |
| 9 (1H) | m | 4.41 | m | 4.38 |
| 9 (1H) | d | 4.46 | d | 4.49 |
| 10 (3H) | s | 1.58 | s | 1.58* |
| 4′(1H) | s | 6.14 | s | 5.86 |
| 1′OH (1H) | s (broad signal) | 12.8 | s (broad signal) | 16.5 |
| 2′ COOH | s (broad signal) | 12.8 | | deprotonation |
| (1H) | | | | |
| 5′ OH (1H) | s | 9.78 | s | 8.80 |
| 1″ (2H) | t (broad signal) | 2.74 | | n.a. |
| 2″ (2H) | m | 1.46 | m | 1.44 |
| 3″,4″ (4H) | m | 1.26 | m | 1.25 |
| 5″ (3H) | t | 0.85 | t | 0.85 |

n.a. . . . not assigned
*peak is overlapped with peak of glucosamine protons

Compared to the pure CBDA, the following changes are observed in the CBDA-GA complex: the signal for 2′ COOH proton is no longer present (likely due to deprotonation), further on, a significant change in the chemical shift for the-OH proton (OH at position 1′) occurs (from δ 12.8 ppm to δ 16.4 ppm). There are also changes in the chemical shifts of the proton of OH group at position 5′ (OH) and proton at position 4′. All listed changes indicate strong interactions between CBDA and GA.

TABLE 8
Characteristic NMR data for CBDA-GA complex.
In the table are listed protons with significant changes
in chemical shifts, compared to the pure CBDA

| H | δ (GA complex) [ppm] | δ (pure) [ppm] |
|---|---|---|
| 1′ (OH) | 16.5 | 12.8 |
| 2′ (COOH) | is not present | 12.8 |
| 4′ (1H) | 5.86 | 6.14 |
| 5′ (OH) | 8.80 | 9.78 |

### 3.4 Hemp Extracts (Resins)

To prove the principle of cannabinoid-GA complex formation also in the case of hemp extracts, 3 different hemp extracts were prepared (EtOH extraction, evaporating the solvent) and modified with glucosamine in the ratio cannabinoids: glucosamine 1:2. NMR spectra of the starting extract and glucosamine modified extracts were recorded. Due to the spectra complexity, only certain protons with characteristic chemical shifts in the complexes were monitored.

### Hemp Extract 1

Among cannabinoids, hemp extract 1 contained predominantly cannabidiol (CBD). The tipical change in the peak shape and slight change in chemical shift occurred for OH protons of CBD at positions 1′ and 5′.

TABLE 9
Characteristic NMR data for CBD resin-GA complex

| | δ (CBD resin-GA | δ (resin) | Difference in peak |
|---|---|---|---|
| H | complex) [ppm] | [ppm] | shape |
| CBD OH protons | 8.68 | 8.67 | peak is broader in the |
| (positions 1′ and | | | CBD resin-GA complex |
| 5′) | | | |

### Hemp Extract 2

Among cannabinoids, hemp extract 2 contained predominantly cannabigerol (CBG) and cannabigerolic acid (CBGA). The tipical change in the peak shape and slight change in chemical shift occurred for OH protons of CBG at positions 1 and 5.

TABLE 10
Characteristic NMR data for CBG resin-GA complex

| | δ (CBG resin-GA | δ (resin) | Difference in peak |
|---|---|---|---|
| H | complex) [ppm] | [ppm] | shape |
| CBG OH protons | 8.94 | 8.89 | peak is broader in the |
| (positions 1 and | | | CBG resin-GA complex |
| 5) | | | |

### Hemp Extract 3

Among cannabinoids, hemp extract 3 contained predominantly cannabidiolic acid (CBDA) and cannabidiol (CBD). The change in chemical shift for proton of OH group at position 1′ (CBDA) was observed, as well the absence of signal for proton of the 2′ COOH group in the 1H NMR spectrum of the complex. Since the extract contains also other diverse compounds, other peaks could not be reliably identified.

TABLE 11
Characteristic NMR data for CBDA resin-GA complex

| | δ (CBDA resin-GA | δ (resin) | Difference in peak |
|---|---|---|---|
| H | complex) [ppm] | [ppm] | shape |
| CBDA COOH | / | 12.1 (broad) | peak is absent due to |
| proton | | | deprotonation |
| (position 2′) | | | |
| CBDA 1′ OH | 16.5 (broad) | 12.1 (broad) | / |
| proton | | | |

### 3.5 Complexes of CBD/GA and CBDA/GA in Different Ratios

NMR spectra were recorded for the complexes CBD/GA and CBDA/GA in the following ratios:
1. CBD/GA complex 1:1
2. CBD/GA complex 1:3
3. CBDA/GA complex 1:1
4. CBDA/GA complex 1:3

Characteristic changes in the chemical shifts were observed in all the analysed samples.

TABLE 12
CBD/GA complexes

| ratio CBD/GA in the | | | | |
|---|---|---|---|---|
| complex | 1:1 | 1:3 | 1:2 | pure CBD |
| δ (OH protons | 8.672 | 8.676 | 8.676 | 8.669 |
| at positions 1′ and 5′) | | | | |
| [ppm] | | | | |

Chemical shifts for the OH protons in the complexes CBD/GA in ratios 1:2 and 1:3 have the same value. Chemical shift in the complex 1:1 is slightly different (lower value), however it is still different from the chemical shift for the mentioned protons in the pure CBD. This is probably the consequence of weaker interactions between CBD and GA in the 1:1 complex, compared to 1:2 and 1:3.

TABLE 13
CBDA/GA complexes

| ratio CBDA/GA in the | | | | pure |
|---|---|---|---|---|
| complex | 1:1 | 1:3 | 1:2 | CBDA |
| δ 1′ (OH) [ppm] | 15.1 | 16.7 | 16.5 | 12.8 |
| δ 2′ (COOH) [ppm] | not present | not | not | 12.8 |
| | | present | present | |
| δ 4′ (1H) [ppm] | 6.01 | 5.84 | 5.86 | 6.14 |
| δ 5′ (OH) [ppm] | 9.08 | 8.73 | 8.80 | 9.78 |

Significant changes in the chemical shifts can be observed in both measured CBDA/GA complexes (1:1 and 1:3) compared to pure CBDA. The changes are most noticeable in the complex in ratio 1:3 and are very similar to the values for the complex 1:2. For the complex in the ratio 1:1 the changes in the values are slightly lower, however still significant. This is as well probably the consequence of weaker interactions between CBDA and GA in the complex 1:1, compared to 1:2 and 1:3 complexes.

### 4. DSC Measurements

All differential-scanning calorimetry (DSC) measurements have been done on a Perkin Elmer Pyris 1 instrument. Measeurements have been conducted in an attempt to determine the eventual (supposed) melting of individual compounds or mixtures or complexes thereof. All measurements have been done in the temperature range between 25 and 110° C. in an inert nitrogen atmosphere (N2 flow 20 ml/min) and consisted of a first heating (at 10° C./min), cooling afterwards (at −1° C./min) and a second heating (also at 10° C./min) immediately after the cooling step.

Physical mixtures of compounds have been made by finely homogenising the compounds in a solid (crystalline) state in a mortar in the same molar ratio (e.g. 1:2) like in the case of complexes, but without »pre-reacting« the compounds in a solution (like in the case of complexes).

The sample containg hemp resin (total CBD cca 30%) which has been complexed with glucosamine has been pre-reacted in solution with 2 molar equivalents of glucosamine (in relation to CBD content) and evaporated.

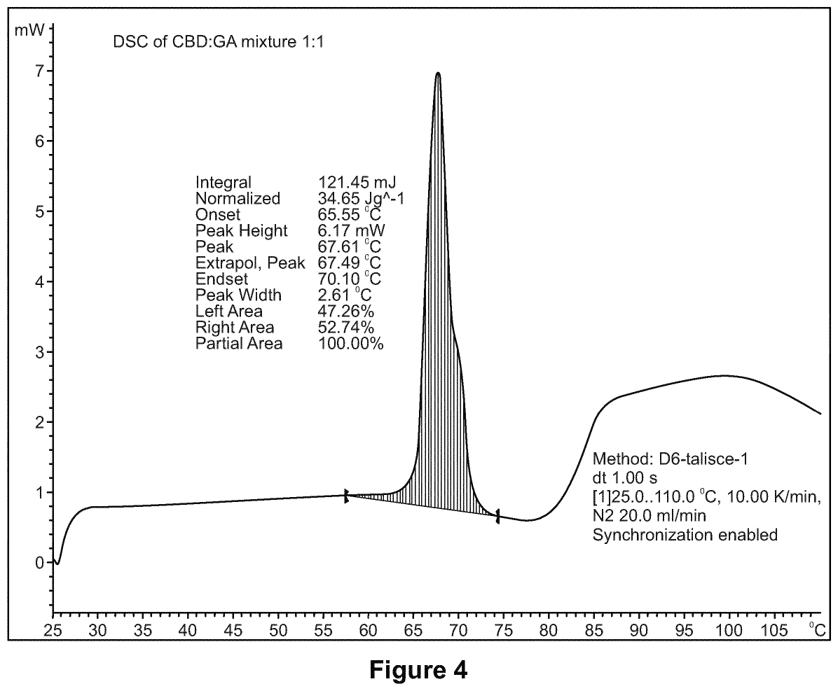

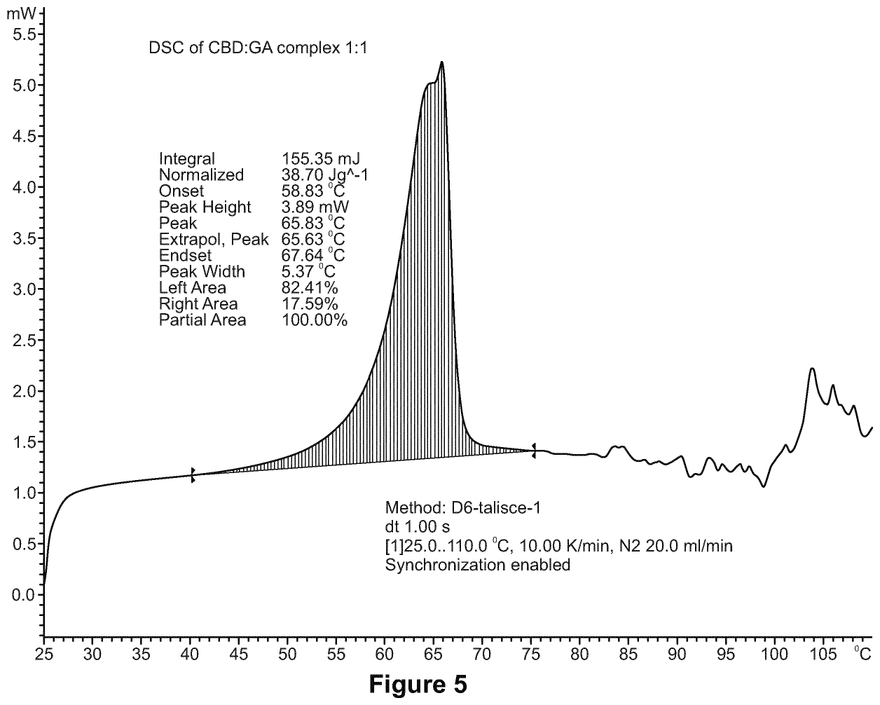

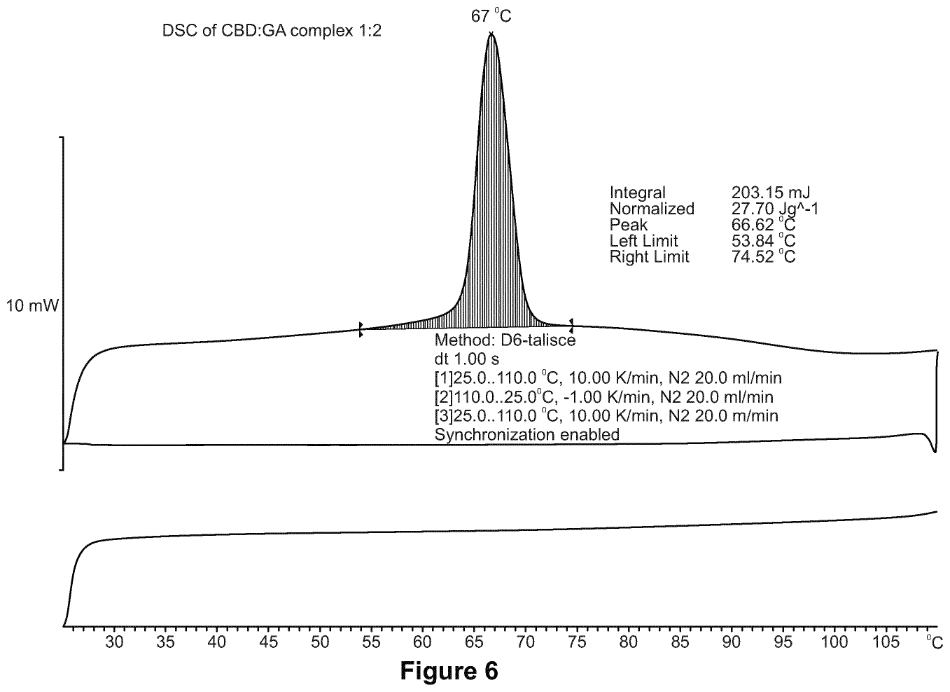

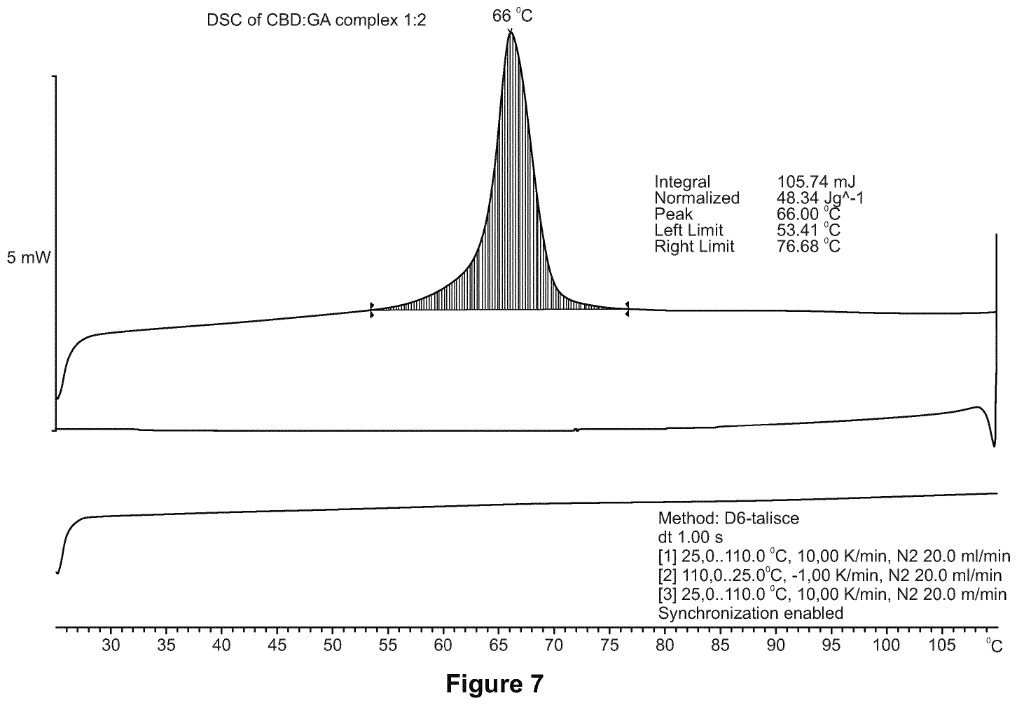

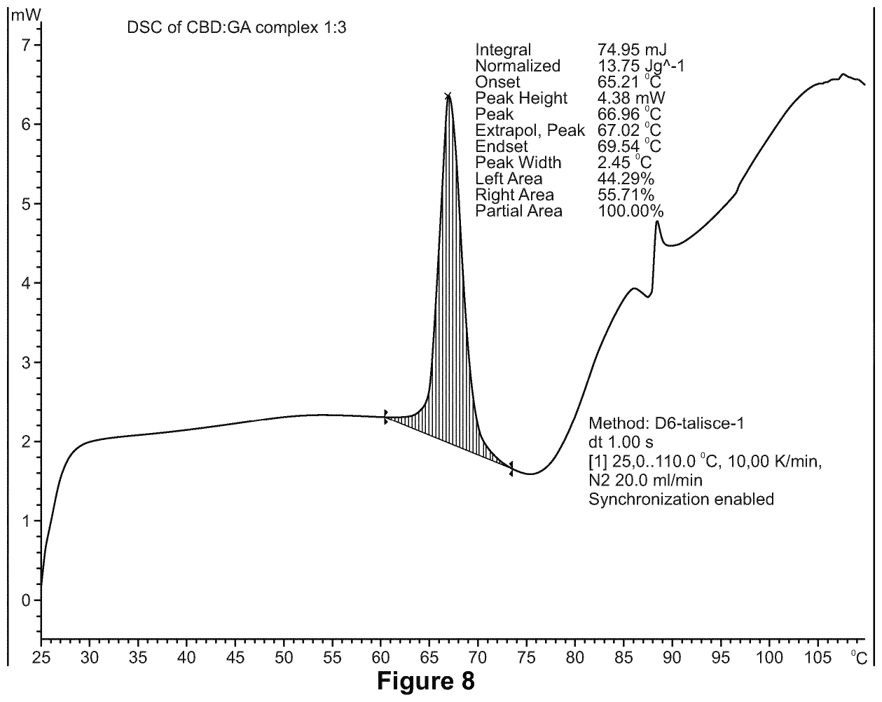

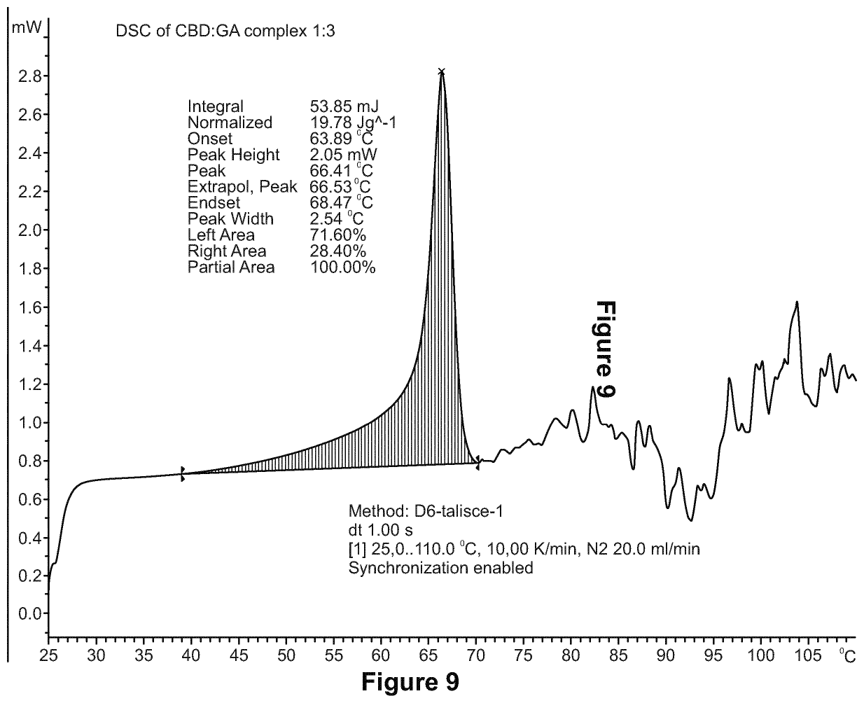

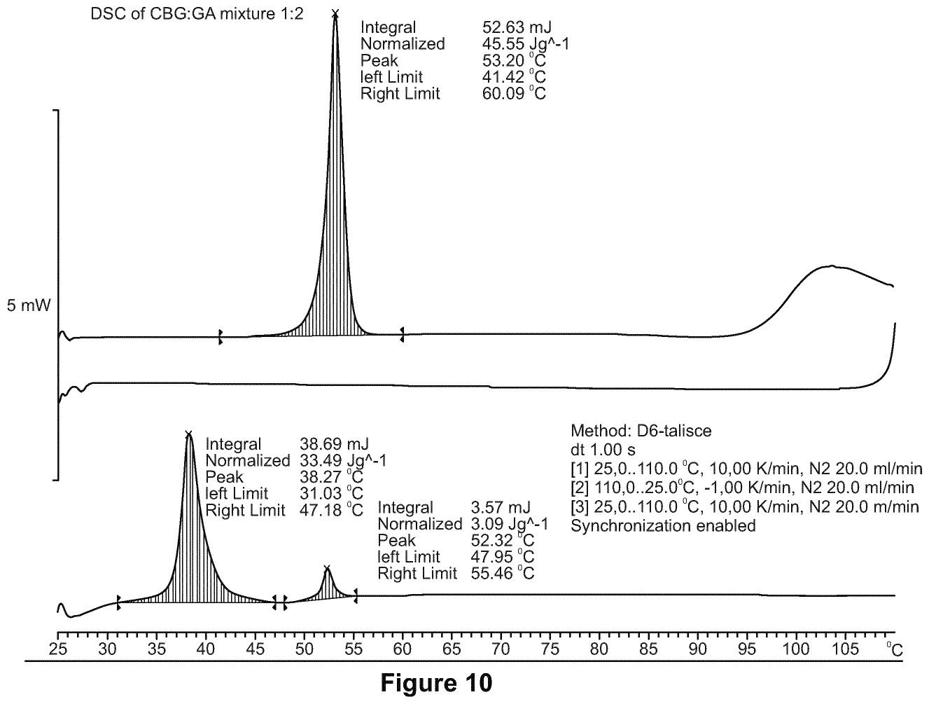

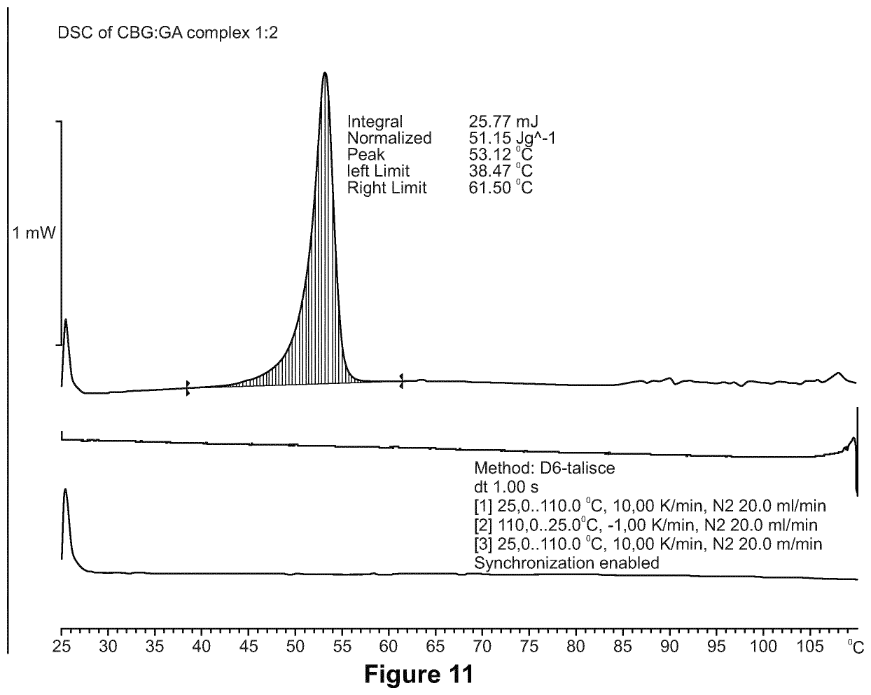

In Table 14 are the collected data obtained from DSC measurements in the form of transition temperature and normalised enthalpy. Exemplary DSC measurements are shown in FIGS. 4 to 11.

TABLE 14
Collected DSC data

| | 1st heating, observed peaks (temp., |
|---|---|
| Sample | enthalpy) |
| CBD | 67.9° C., 62.7 J/g |
| CBG | 53.4° C., 119.1 J/g |
| CBDA | 90.1° C., −0.06 J/g; 102.6° C., −0.04 J/g |
| Glucosamine (GA) | 84.1° C. (very wide), 42.1 J/g |
| CBD + GA 1:1 mixture | 67.6° C., 34.7 J/g |
| CBD + GA 1:1 complex | 65.8° C., 38.7 J/g |
| CBD + GA 1:2 mixture | 66.6° C., 27.7 J/g |
| CBD + GA 1:2 complex | 66.0° C., 48.3 J/g |
| CBD + GA 1:3 mixture | 67.0° C., 13.8 J/g |
| CBD + GA 1:3 complex | 66.4° C., 19.8 J/g |
| CBG + GA 1:2 mixture | 53.2° C., 45.6 J/g |
| CBG + GA 1:2 complex | 53.1° C., 51.2 J/g |
| CBDA + GA 1:2 complex | 73.6° C. (very wide), 38.4 J/g; |
| | 104.6° C., 0.31 J/g; 107.4° C., 0.37 J/g |
| Resin | none |
| resin + GA complex | 78.1° C. (very wide), 13.8 J/g |

It is demonstrated that complexes have a different behaviour compared to physical mixtures or pure compounds, since differences are observed in transition temperatures and/or their enthalpies.

### 5. ATR-FTIR Measurements

Measurements of Attenuated Total Reflectance Fourier-Transform Infrared (ATR-FTIR) spectra were performed at 298 K on the Bruker Tensor 27 spectrometer using a single reflectance Specac Golden Gate diamond ATR accessory. A nitrogen-cooled MCT detector was used, and the spectrometer optics and ATR cell were sealed from the atmosphere and purged with technical dry nitrogen during measurements. ATR-FTIR spectra were recorded in the range 4000 cm⁻¹ to 600 cm⁻¹. 128 scans were averaged with a nominal resolution of 2 cm⁻¹. The temperature was controlled using a Specac Heated Golden Gate Controller. The surface of the diamond ATR crystal was cleaned with acetone prior to application of each sample and measurement. Water and CO₂ atmospheric compensation was applied to the obtained spectra.

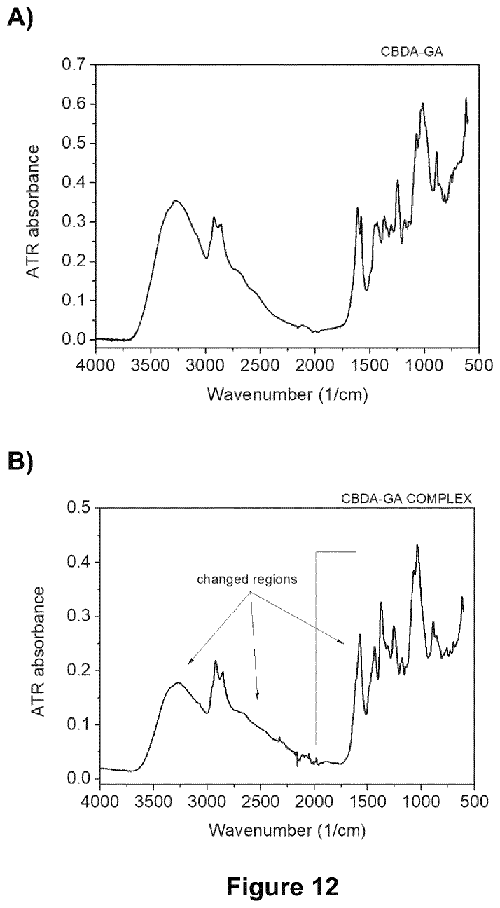

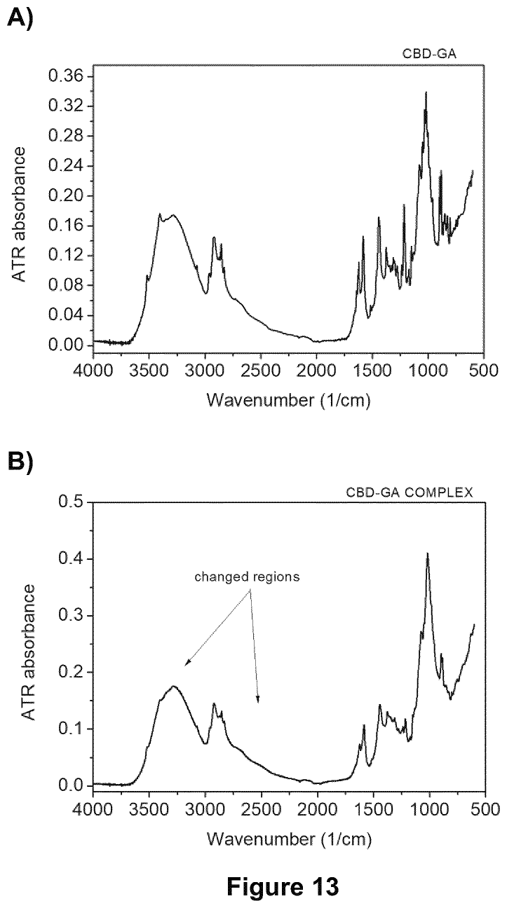

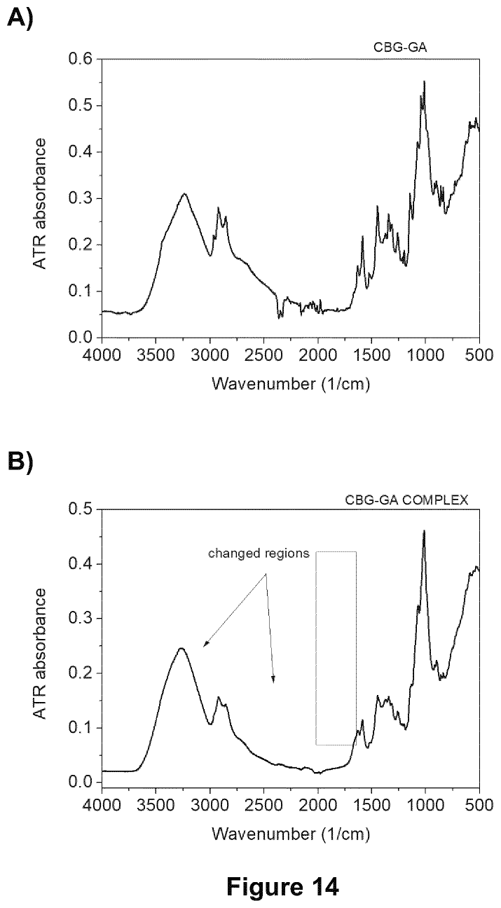

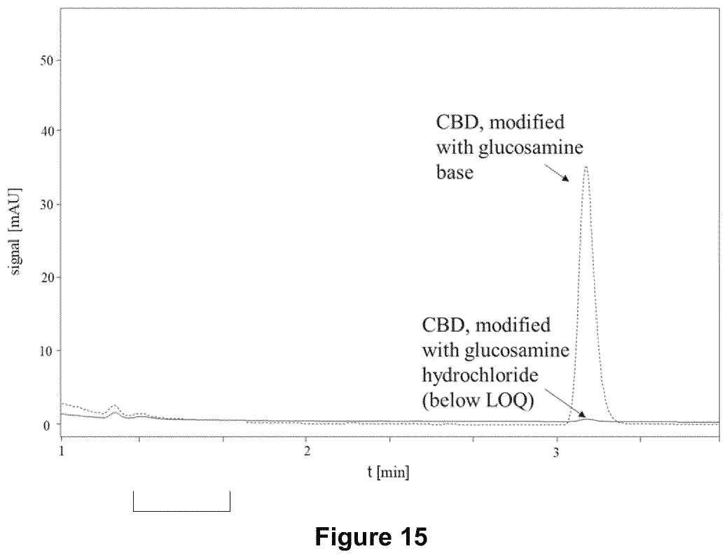

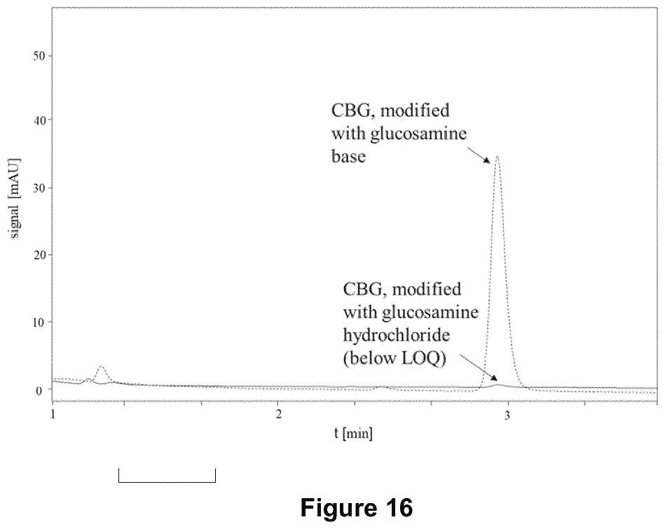

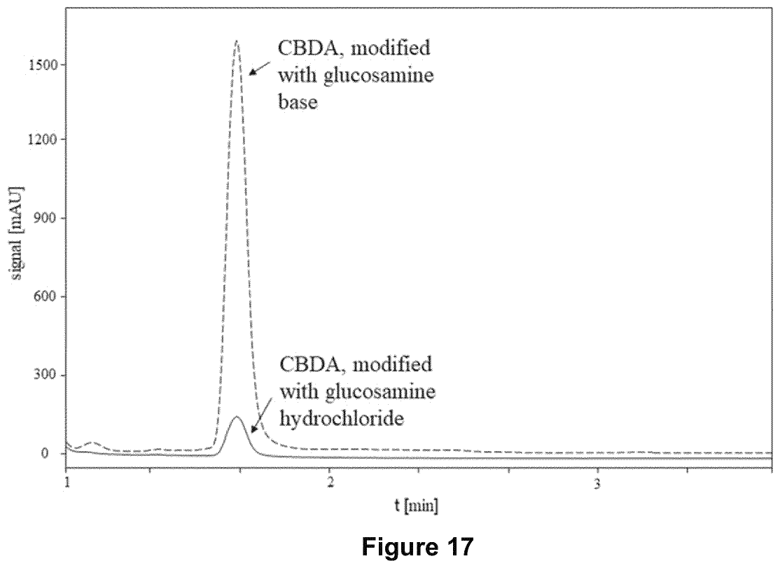

The spectra of physical mixtures are shown in FIGS. 12A, 13A and 14A. It is evident for all three cases that the ATR spectra of the mixtures correspond to the sum of the spectra of the pure components. No significant change in frequencies and/or band shape is observed in these spectra. This means that no new interactions develop between the components of the mixtures that are not already present in the pure components.

The spectra of the complexes are shown in FIGS. 12B, 13B and 14B. These spectra are quite different from those of the pure components and those of the mixture. It is therefore evident that the complexation has destroyed some of the pre-existing hydrogen bonds and established new connectivity. The spectral regions with the most striking changes are shown in the figures of the complexes. The strongest changes were observed in the CDBA-GA complexes, while the other two complexes showed smaller spectral changes. The reason for this lies in the particular group that is unique to the CDBA molecule and belongs to the carboxylic COOH. This group acts both as a very efficient proton donor and proton acceptor and therefore tends to form new modified hydrogen bonds when in the complex GA molecule is present.

### LIST OF CERTAIN REFERENCES CITED IN THE DESCRIPTION

Anderson, L. L.; Low, I. K.; Banister, S. D.; McGregor, I. S.; Arnold, J. C. Pharmacokinetics of phytocannabinoid acids and anticonvulsant effect of cannabidiolic acid in a mouse model of Dravet syndrome. J. Nat. Prod. 2019, 82, 3047-3055.
Bolognini, D.; Rock, E. M.; Cluny, N. L.; Cascio, M. G.; Limebeer, C. L.; Duncan, M.; Stott, C. G.; Javid, F. A.; Parker, L. A.; Pertwee, R. G. Cannabidiolic acid prevents vomiting in Suncus murinus and nausea-induced behaviour in rats by enhancing 5-HT1A receptor activation. Br. J. Pharmacol. 2013, 168, 1456-1470.
Shu, C-K. Degradation Products Formed from Glucosamine in Water. J. Agric. Food Chem. 1998, 46, 1129-1131.
Dolci, M., Pereira L., Edge, T. Hydrophilic Interaction Liquid Chromatography: Method Development Approaches. 2014, https://www.eposters.net/pdfs/hydrophilic-interaction-liquid-chromatography-method-development-approaches-. pdf
Izgelov, D.; Domb, A. J.; Hoffman, A. The effect of piperine on oral absorption of cannabidiol following acute vs. chronic administration. Eur. J. Pharm. Sci. 2020, 148, 105313.
Koch, N.; Jennotte, O.; Gasparrini, Y.; Vandenbroucke, F.; Lechanteur, A.; Evrard, B. Cannabidiol aqueous solubility enhancement: Comparison of three amorphous formulations strategies using different type of polymers. Int. J. Pharm. 2020, 589, 119812.
Križman, M. A simplified approach for isocratic HPLC analysis of cannabinoids by fine tuning chromatographic selectivity. Eur. Food. Res. Technol. 2020, 246, 315-322.
Mechoulam, R.; Ben-Zvi, Z. Carboxylation of resorcinols with methylmagnesium carbonate. Synthesis of cannabinoid acids. J. Chem. Soc. D Chem. Comm. 1969, 7, 343-344.
Pugazhendhi, A.; Suganthy, N.; Chau, T. P.; Sharma, A.; Unpaprom Y.; Ramaraj, R.; Karuppusamy I.; Brindhadevi, K. Cannabinoids as anticancer and neuroprotective drugs: Structural insights and pharmacological interactions—A review. Process Biochem. 2021, 111, 9-31.
Stukelj, R.; Bencina, M.; Faretti, M.; Valant, M.; Drab, M.; Iglic, A.; Kralj-Iglic, V. Mater. Technol. 2019, 53, 543-549.
Takeda, S.; Misawa, K.; Yamamoto, I.; Watanabe, K. Cannabidiolic acid as a selective cyclooxygenase-2 inhibitory component in cannabis. Drug Metab. Dispos. 2008, 36, 1917-1921.
Takeda, S.; Okazaki, H.; Ikeda, E.; Abe, S.; Yoshioka, Y.; Watanabe, K.; Aramaki, H. Down-regulation of cyclooxygenase-2 (COX-2) by cannabidiolic acid in human breast cancer cells. J. Toxicol. Sci. 2014, 39, 711-716.

1. Complex comprising a) a cannabinoid and b) glucosamine.

2. The complex according to claim 1, wherein the molar ratio between the cannabinoid and glucosamine is ranging from 1:0.5 to 1:15.

3. The complex according to claim 1, wherein the molar ratio between the cannabinoid and glucosamine is ranging from 1:1 to 1:5.

4. The complex according to claim 1, wherein the molar ratio between the cannabinoid and glucosamine is 1:1.

5. The complex according to claim 1, wherein the molar ratio between the cannabinoid and glucosamine is 1:2.

6. The complex according to claim 1, wherein the molar ratio between the cannabinoid and glucosamine is 1:3.

7. The complex according to claim 1, wherein the cannabinoid is selected from the group consisting of: cannabinol (CBN), cannabinolic acid (CBNA), cannabidiol (CBD), cannabidiolic acid (CBDA), cannabigerol (CBG), cannabigerolic acid (CBGA), tetrahydrocannabinol (THC), tetrahydrocannabinolic acid (THCA), Δ(9)-tetrahydrocannabinol (D9THC), Δ(9)-tetrahydrocannabinolic acid (D9THCA), Δ(9)-cannabidiol (D9CBD), Δ(9)-tetrahydrocannabidiolic acid, (D9THCA), Δ(8)-tetrahydrocannabinol (D8THC), Δ(8)-tetrahydrocannabinolic acid (D8THCA), tetrahydrocannabivarin (THCV), tetrahydrocannabivarin (THCV), tetrahydrocannabivarinic acid (THCVA), cannabichromene (CBC), cannabichromenic acid (CBCA), cannabicyclol (CBL), cannabicyclolic acid (CBLA), cannabielsoin (CBE), cannabielsoic acid (CBEA), cannabitriol (CBT), cannabitriolic acid (CBTA), Nabilone, and combinations thereof.

8. The complex according to claim 1, wherein the cannabinoid is selected from the group consisting of cannabinol, cannabinolic acid, cannabidiol (CBD), cannabidiolic acid (CBDA), cannabigerol, cannabigerolic acid, and combinations thereof.

9. The complex according to claim 1, wherein the cannabinoid is selected from the group consisting of cannabidiol (CBD), cannabidiolic acid (CBDA), cannabigerol (CBG), cannabigerolic acid (CBGA), and combinations thereof.

10. The complex according to claim 1, wherein the cannabinoid is selected from the group consisting of cannabidiol (CBD), cannabidiolic acid (CBDA), and a combination thereof.

11. The complex according to claim 1, wherein the solubility of the cannabinoid in water is at least about 15 mg/L.

12. The complex according to claim 1, wherein the solubility of the cannabinoid in water is in the range from about 15 mg/L to about 110 mg/L.

13. The complex according to claim 1, wherein the cannabinoid is cannabidiolic acid (CBDA).

14. The complex according to claim 13, wherein the solubility of the cannabinoid in water is at least about 30 mg/L or at least about 75 mg/L.

15. The complex according to claim 1, wherein the cannabinoid is cannabigerol (CBG).

16. The complex according to claim 1, wherein said complex is an isolated complex.

17. A composition comprising a) a cannabinoid and b) glucosamine, wherein the solubility of the cannabinoid in water is at least about 15 mg/L.

18. The composition according to claim 17, wherein the solubility of the cannabinoid in water is in the range from about 15 mg/L to about 110 mg/L.

19. The composition according to claim 17, comprising at least one complex comprising a cannabinoid and glucosamine.

20. Process for the preparation of a complex according to claim 1, said process comprising reacting the cannabinoid with glucosamine base.

21. Method for improving the solubility of a cannabinoid in water, comprising the step of reacting the cannabinoid with glucosamine base.

22. The process according to claim 20, further comprising a pre-treatment step which comprises stripping off a counter ion from glucosamine in salt form to obtain said glucosamine base.

23. The process according to claim 20, wherein the step of reacting the cannabinoid with glucosamine base is followed by a concentration step.

24. (canceled)

25. The process according to claim 23, wherein the concentration step is evaporation of a solvent.
